(19) United States
(12) Patent Application Publication
(10) Pub. No.: US 2025/0115936 A1
(43) Pub. Date: Apr. 10, 2025

(54) PRODUCTION OF XYLITOL FROM A MIXTURE OF HEMICELLULOSIC SUGARS

(71) Applicants: THE BOARD OF TRUSTEES OF THE UNIVERSITY OF ILLINOIS, Urbana, IL (US); ZUCHEM INC., Chicago, IL (US)

(72) Inventors: Huimin Zhao, Champaign, IL (US); Nikhil Unni Nair, Urbana, IL (US); Michael Racine, Peoria, IL (US); Ryan Woodyer, Normal, IL (US)

(21) Appl. No.: 18/666,259

(22) Filed: May 16, 2024

Related U.S. Application Data
(63) Continuation of application No. 17/116,704, filed on Dec. 9, 2020, now abandoned.
Application No. 17/116,704 is a continuation of application No. 16/167,207, filed on Oct. 22, 2018, now abandoned.
Application No. 16/167,207 is a continuation of application No. 13/521,366, filed on Jul. 10, 2012, now Pat. No. 10,106,824.
(60) Provisional application No. 61/295,551, filed on Jan. 15, 2010.
(60) Provisional application No. 61/328,609, filed on Apr. 27, 2010.
(60) Provisional application No. 61/391,951, filed on Oct. 11, 2010.

(51) Int. Cl. C12P 7/18

(52) U.S. Cl. CPC C12P 7/18

(57) ABSTRACT

Materials and methods are described to produce xylitol from a mixture of hemicellulosic sugars by several routes. Examples include either as a direct co-product of a biorefinery or ethanol facility, or as a stand-alone product produced from an agricultural or forestry biomass feedstock including using, e.g. ethanol waste streams.

### CROSS REFERENCE TO RELATED APPLICATIONS

The present application is a continuation of U.S. patent application Ser. No. 16/167,207, filed Oct. 22, 2018, which is a continuation of U.S. patent application Ser. No. 13/521,366, filed Jul. 10, 2012, now U.S. Pat. No. 10,106,824, which is a U.S. National Phase Application pursuant to 35 U.S.C. § 371 of International Application No. PCT/US 11/21277 filed Jan. 14, 2011, which claims priority to U.S. Application No. 61/295,551, filed Jan. 15, 2010, Application No. 61/328,609, filed Apr. 27, 2010, and Application No. 61/391,951, filed Oct. 11, 2010. The disclosures set forth in the referenced applications are incorporated herein by reference in their entireties, including all information as originally submitted to the United States Patent and Trademark Office.

### SEQUENCE LISTING STATEMENT

A computer readable form of the Sequence Listing is filed with this application by electronic submission and is incorporated into this application by reference in its entirety. The Sequence Listing is contained in the file created on Jan. 24, 2021, having the file name “14-473-US-CON2_Corrected Sequence-Listing_ST25.txt” and is 6,500 bytes in size.

### BACKGROUND

Materials and methods are described to produce xylitol from a mixture of hemicellulosic sugars by several routes. Examples include either as a direct co-product of a biorefinery or ethanol facility, or as a stand-alone product produced from an agricultural or forestry biomass feedstock including using, e.g. ethanol waste streams.

Xylitol has several favorable properties as a sugar substitute, such as low caloric content, anticariogenicity, good gastrointestinal tolerance, and near insulin-independent metabolism in humans. The traditional production of xylitol involves direct chemical hydrogenation of hemicellulosic hydrolysates over a Raney-Nickel catalyst followed by extensive purification from non-specific reduction products. In the chemical process, D-xylose is converted to xylitol by catalytic reduction. This method utilizes specialized and expensive equipment for the high pressure and temperature requirements as well as the use of a Raney-Nickel catalyst that can introduce trace nickel into the final product, which is undesirable. Additionally, the overall yield is only 50-60%. The final product must also be purified. This multi-step process is expensive and inefficient.

Hydrolysate from birch trees has historically been the only economic source of xylose used to make xylitol by chemical hydrogenation. Birch tree hydrolysate is a byproduct of the paper and pulping industry and it has only minor amounts of arabinose and other sugars. However availability severely limits this source of xylitol. Hydrolysis of other xylan-rich materials, such as trees, straws, corncobs, oat hulls under alkaline conditions also yields hemicellulose hydrolysate, however these hydrolysates contain too many sugars other than xylose, especially L-arabinose. These competing sugars create a number of by-products during the hydrogenation process that are difficult and costly to remove.

Biocatalytic routes to xylitol production using fungal or yeast xylose reductase (XR) have also been explored. Unfortunately, the nonspecific nature of direct hydrogenation is only partially addressed in the biocatalytic route. The natural promiscuity of XRs toward other sugars, particularly L-arabinose, another major component of hemicelluloses, necessitates the prior purification of D-xylose to minimize formation of L-arabinitol. Because D-xylose and L-arabinose are epimers, their separation is nontrivial, and is one of the leading obstacles to the more economical production of xylitol.

Because there is a significant amount of arabinose in the hydrolysates, a significant amount of arabinitol (arabitol) is produced because the xylose reductase enzyme that converts xylose to xylitol also converts arabinose to arabinitol. A significant challenge was to develop either a process that produces negligible amounts of arabinitol or alternatively converts the arabinose into additional xylitol.

While some basic research has been performed by others in the field, development of an effective bioprocess for the production of xylitol has been elusive. Many of these systems suffered from problems such as poor microbial strain performance, low volumetric productivity, and too broad of a substrate range. Moreover, kinetics and overall performance of the enzymes reported to date have not been engineered (via methods such as directed evolution) to maximize efficiency. More efficient enzyme activity would result in improved throughput and shorter reaction times, both of which are crucial to a commercially viable process.

Most of the research performed has also been carried out using a highly purified and concentrated D-xylose substrate. This substrate has no significant amounts of other pentoses such as arabinose or other hexoses such as D-glucose. While some reasonable yields with such a substrate have been reported, developing a bioprocess with pure D-xylose is impractical due to the cost of this substrate and the fact that it can be hydrogenated at similar costs and better space-time yields.

None of the approaches described in this section have been commercially effective for a number of reasons. First, xylose uptake is often naturally inhibited by the presence of glucose that is used as a preferred carbon source for many organisms. Second, none of the enzymes involved have been optimized to the point of being cost effective. Finally, xylose in its pure form is expensive and any requirement for a bioprocess to use pure xylose results in direct competition with inexpensive chemical hydrogenation. Additionally, all of the systems developed would produce arabinitol as a significant contaminating byproduct since the xylitol dehydrogenase used has similar activity with both xylose and arabinose.

Xylitol could potentially be a byproduct of ethanol production. When products such as ethanol or other chemicals are produced from corn by current processes, only starch is generally utilized. Thus, during ethanol production, significant by-products rich in pentose and other sugars are made. For example, when ethanol is produced from a dry-mill operation (about 55% of the facilities today) distiller's dry grains (DDG) and other byproducts are produced. In the wet-mill operation (the remaining 45% of current facilities) corn fiber rich in hemicellulose is produced. These products are usually sold as inexpensive animal feed or otherwise disposed of, but both the corn fiber and distiller's dry grains could potentially be converted to other value-added products, such as xylitol which could help improve the economics of ethanol production.

### SUMMARY OF THE DISCLOSURE

Methods and compositions are disclosed to produce xylitol—some that are useful on an industrial scale, and all having advantages. Methods include a new process that would allow xylitol to be produced from a variety of agricultural and forestry derived hemicellulose feedstocks such as hardwoods, softwoods, bagasse, wheat straw, corn and corn fiber, sources such as those that are leftover from U.S. ethanol production, bioenergy production, or other biochemical production. Fermentation organisms were designed to alleviate some of the previous problems, notably by minimizing arabinitol.

A variety of fermentation systems disclosed herein are able to convert a hemicellulose mixture (arabinose, xylose, and a variety of C6 sugars) to a low-arabinitol product.

Systems to produce xylitol include:
(A) conversion of xylose to xylitol by a xylose reductase;
(B) conversion of L-arabinose to xylitol, reduce xylose;
(C) reduce D-xylose and metabolize arabinose.

Aspects of the invention also include:
(A) preparation and improvement of industrial hemicellulose samples;
(B) analysis of fermentation inhibition by different industrial hemicellulose samples; and
(C) novel xylose reductase genes.

Aspects of this disclosure include an E. coli strain that efficiently produces xylitol from D-xylose, wherein xylitol is produced at a purity of approximately 90-100% from an equivalent mixture of D-xylose, L-arabinose, and D-glucose. The method to reduce D-xylose to xylitol uses an engineered E. coli strain, wherein there is a minimal production of L-arabinitol byproduct.

The biocatalytic reduction of D-xylose to xylitol requires separation of the substrate from L-arabinose, another major component of hemicellulosic hydrolysate. This step is necessitated by the innate promiscuity of xylose reductases, which can efficiently reduce L-arabinose to L-arabinitol, an unwanted byproduct. Unfortunately, due to the epimeric nature of D-xylose and L-arabinose, separation can be difficult, leading to high production costs. To overcome this issue, an E. coli strain is disclosed that efficiently produces xylitol from D-xylose with minimal production of L-arabinitol byproduct. By combining this strain with a previously engineered xylose reductase mutant, (SEQ ID NO: 19 and 20) L-arabinitol formation is eliminated and xylitol is produced to near 100% purity from an equiweight mixture of D-xylose, L-arabinose, and D-glucose.

### BRIEF DESCRIPTION OF THE DRAWINGS

FIG. 1. Potential pathways for converting xylose or arabinose to xylitol. (A) Pathways A—Conversion of xylose to xylitol via xylose reductase; (B) Pathway B—conversion of xylose to xylitol via a D-xylulose intermediate; (C) Pathway C; conversion of arabinose to xylitol via epimerase.

FIG. 2: Conversion of xylose to xylitol via xylose reductase: (A) C. globosum (SEQ ID NO: 2); (B) N. crassa (SEQ ID NO: 19 and 20); (C) and (D) bioconversion with the XR from (A) and (B).

FIG. 3: Conversion of C-5 mixed sugars to xylitol; via a D-xylulose intermediate (Xl/XDH): (A) pATX210, (B) L-arabinose to xylitol.

FIG. 3C: Conversion of C-5 mixed sugars to xylitol; via a D-xylulose intermediate (Xl/XDH): pATX215.

FIG. 4: Conversion of L-arabinose in to xylitol by the epimerase of Pathway C.

FIG. 5: Two stage production of xylitol in biomass hydrolysate using first the L-arabinose to xylitol (epimerase) Pathway C followed by the xylose to xylitol (xylose reductase) Pathway A: (A) 2 stage, (B) 2 stage higher sugars.

FIG. 6: Conversion of a C-5 mixture to xylitol and arabinitol with ZUC138 (A) containing a plasmid with combined genes for Pathway A and Pathway C (pATX221); and (B) bioconversion to xylitol and arabitol.

FIG. 7: Conversion of a C-5 mixture to xylitol with ZUC142 (A) containing a plasmid with combined genes for Pathway B and Pathway C (pATX231); and (B) bioconversion to xylitol and arabitol.

FIG. 8: Production of xylitol from biomass hydrolysate in a single stage bioconversion using an Ara+ Strain.

FIG. 9: Efficient conversion of biomass hydrolysate to xylitol with low production of arabitol: (A) corn fiber, (B) hardwood.

FIG. 10. Growth of various strains in D-glucose, D-xylose, and L-arabinose to test for catabolite repression at 30° C. (A) Wild-type E. coli K-12 C600 shows strong diauxie, with quick utilization of D-glucose first. (B) Deletion of the regulatory domain of adenylate cyclase (HZ1651, ΔcyaA^{regul}) resulted in slightly less pronounced diauxie, although pentose assimilation is still slower than D-glucose. (C) D-Glucose permease knockout (HZ1743, ΔptsG) strain showed efficient L-arabinose and D-glucose utilization, although D-xylose was relatively slower. (D) The mutant CRP (HZ1302, crp*) showed the most efficient co-utilization of all three sugars. All experiments were also performed at 37° C. to ascertain D-glucose de-repressed phenotype.

FIG. 11. Xylitol production in shake flasks comparing (A) HZ1757 (ΔptsG ΔxylA pXXR) and (B) HZ1434 (crp*ΔxylA pXXR SEQ ID NO: 6) diauxie relief strategies. Although both strains demonstrate simultaneous glucose and L-arabinose assimilation, stronger induction of the xylose pathway results in higher xylitol production using XR under XylA promoter in HZ1434. Neither of the two strains produces significant amounts of L-arabinitol. Data are an average of two independent experiments and error is less than 15% in all cases. Experiments were also performed with mutant VMQCI, (SEQ ID NO: 19 and 20) and similar results were obtained.

FIG. 12. Strategies implemented to improve xylitol productivity. (A) pH-stat bioreactor allows cells to completely and efficiently catabolize L-arabinose and glucose simultaneously. XR expression is under control of the XylA promoter (HZ1434). (B) Concurrent expression of xylose-proton symporter (XylE) using AraBAD promoter decreases lag phase, but also decreases L-arabinose assimilation rate relative to glucose (HZ2008). Xylitol productivity does not increase significantly, however. (C) Expression of XR using AraBAD promoter instead of XylA promoter promotes near-stoichiometric conversion of D-xylose to xylitol (HZ2061). (D) Expression of the mutant XR, VMQCI, eliminates L-arabinitol production, although initial xylitol productivity also drops slightly (HZ2062). Data are an average of two independent experiments and error is less than 15% in all cases.

FIG. 13. Acetate production by HZ1434 during growth in 4% and 0.4% usable sugars (glucose+L-arabinose). Cells grown in high concentrations of sugars succumb to Crabtree effect and produce large amounts of acetate (˜25 mM), which inhibits cell growth, resulting in decreased final cell density. Data points are shown at 0, 6, 24, 48 h, and are an average of two independent experiments and error is less than 15% in all cases.

FIG. 14. ZUC220 with synthetic hydrolysate.

FIG. 15. Action of ZUC170 on corn fiber hydrolysate with fermenter to fermenter transfer.

FIG. 16. Gene sequence of xylose reductase from Chaetomium globosum (SEQ ID NOS 1-2, respectively, in order of appearance).

FIG. 17. Xylitol recovery.

FIG. 18. NcXRwt sequence (SEQ ID NOS 3-4, respectively, in order of appearance).

FIG. 19. pACYC-ncxr sequence (SEQ ID NO: 5).

FIG. 20. pXXR sequence (SEQ ID NO: 6).

FIG. 21. pTrcXR sequence (SEQ ID NO: 7).

FIG. 22. pAraXR sequence (SEQ ID NO: 8).

FIG. 23. NcXR mutant S sequence (SEQ ID NOS 9-10, respectively, in order of appearance).

FIG. 24. NcXR mutant Q sequence (SEQ ID NOS 11-12, respectively, in order of appearance).

FIG. 25. NcXR mutant QC sequence (SEQ ID NOS 13-14, respectively, in order of appearance).

FIG. 26. NcXR mutant MQC sequence (SEQ ID NOS 15-16, respectively, in order of appearance).

FIG. 27. NcXR mutant MQCI sequence (SEQ ID NOS 17-18, respectively, in order of appearance).

FIG. 28. NcXR mutant VMQCI sequence (SEQ ID NOS 19-20, respectively, in order of appearance).

FIG. 29. pACYCAra XylE sequence (SEQ ID NO: 21).

FIG. 30. Strain derivations.

FIG. 31. Diagram of development of HZ strains (see Table 1).

### DETAILED DESCRIPTION

I. Materials and methods are described to produce xylitol by several routes for example either as a direct co-product of a biorefinery or ethanol facility, or produced as a stand-alone product using, e.g. ethanol waste streams.

### A. Conversion of Xylose to Xylitol Via Xylose Reductase.

### 1. Use D-Xylose Reductase on Arabinose-Depleted Feedstock; in an Arabinose Utilizing Organisms; Xylose Reductases Will Reduce Arabinose:

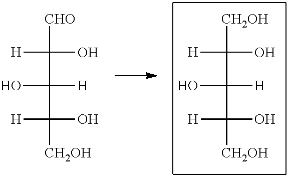 [SMILES: O=CC(O)C(O)C(O)CO ; OCC(O)C(O)C(O)CO]

Two organisms designated ZUC140 and ZUC166 can accomplish this. Xylose can be converted to xylitol at a high efficiency, but also produces arabitol from arabinose (tested at 50:50 ratio).

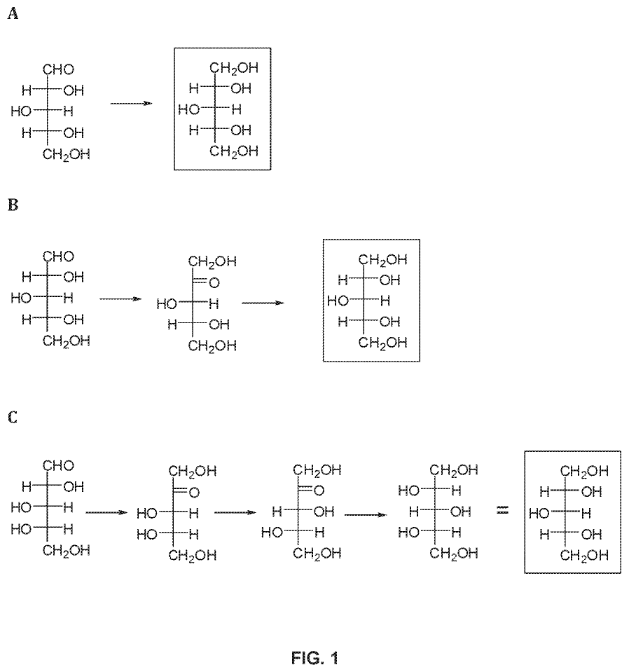

One way to convert xylose to xylitol is directly through the use of a xylose reductase as depicted in FIG. 1A (Pathway A). Several xylose reductase genes had previously been cloned into E. coli expression vectors, expressed, and tested for ability to convert xylose into xylitol. Most genes are expressed and very active in constitutive expression systems within strain ZUC134. E. coli strain Zuc134 was created from K12 prototroph AB707 through a combination of PCR based genetic deletion and selection for improved growth on glucose. First ptsG was removed, followed by xylB, then araBAD, and finally lyxK in successive order. The final strain was then selected on M9+glucose liquid medium several times for improved growth, a single colony was isolated, cultured, and stored at −80° C.

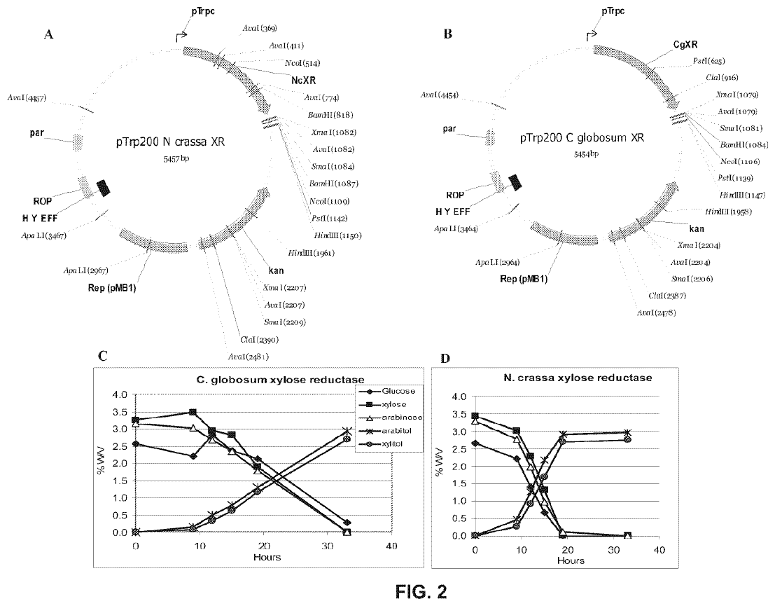

The best results achieved were with the xylose reductases from Neurospora crassa (McXR) and Chaetomium globosum (CgXR) [FIG. 2(D), (C)]. CgXR was synthetically constructed for E. coli expression [FIG. 2(A)], whereas NcXR was isolated from mRNA of N. crassa [FIG. 2B]. Both genes were placed in the expression vector pTRP200 under the pTRP promoter allowing constitutive expression. The resulting strains ZUC140 (ZUC134 NcXR) and ZUC166 (ZUC134 CgXR) are very powerful reducing biocatalysts.

The ability to convert a “synthetic hemicellulose” mixture that contained both xylose and arabinose together as a starting material was investigated. Although hemicelluloses vary in concentration of these sugars, a 50:50 mixture was used in these experiments, unless otherwise indicated. This can be supplemented by an additional C6 sugar such as glucose for growth of the strains.

One liter bioconversions were performed to test these systems with a synthetic hemicellulose substrate containing a 50:50 mixture of xylose and arabinose (30 g/L each). In these experiments WC140 was capable of reducing 30 g D-xylose to xylitol in just 20 hrs. ZUC166 was capable of the same reduction in approximately 30 hrs. Both of these systems, however, concurrently reduce 30 g L-arabinose to L-arabitol over the same time period. A problem is that L-arabitol is an undesirable side product. Method: 2 L BiostatB (Sartorius). Medium: 10 g/L tryptone, 5 g/L yeast extract, 5 g/L sodium chloride; 2.6 g/L dibasic potassium phosphate Sterilized in 800 mL. Sugars sterilized separately and added in 150 mL prior to inoculation to indicated starting concentrations. [glucose was also added, same concentration, not shown]. Inoculated with 50 mL seed, overnight shake flask in LB medium. pH autocontrolled at pH 7.0 with ammonium hydroxide, Temp 30° C., agitation 800 rpm, 1 vvm air (1 Lpm). Products were tested by HPLC.

2. Conversion of C-5 Mixed Sugars to Xylitol Via a n-Xylulose Intermediate (XIIXDH).

Isomerize D-xylose to D-xylulose; reduce D-xylulose to xylitol (arabinose is unaffected by either enzyme):

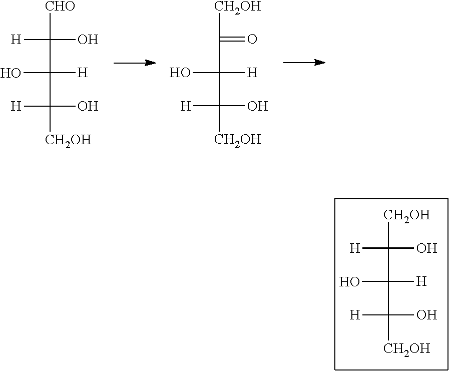 [SMILES: O=CC(O)C(O)C(O)CO ; O=C(CO)C(O)C(O)CO ; OCC(O)C(O)C(O)CO]

Another method to convert xylose to xylitol has the advantage of not converting L-arabinose to arabitol, because both enzymes (XI and XDH) do not have any activity with L-arabinose as depicted in FIG. 1B (Pathway B). Plasmid pZUC036 (see U.S. 2006/0110809 incorporated herein by reference) contained a XI cloned from E. coli and a XDH cloned from Trichoderma reesei (Hypocrea jecorina) under the control of the pTRP constitutive promoter.

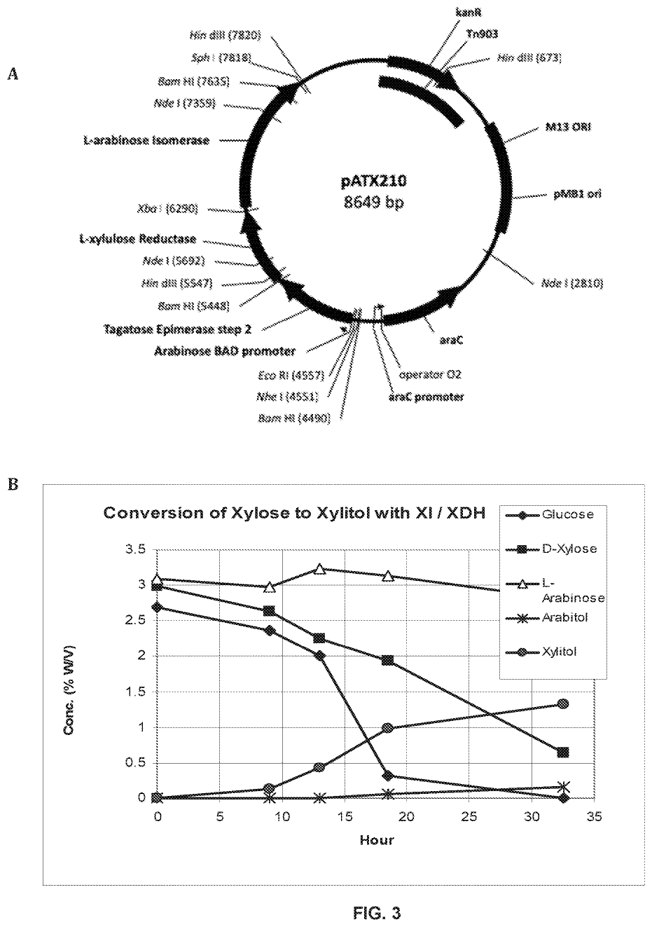

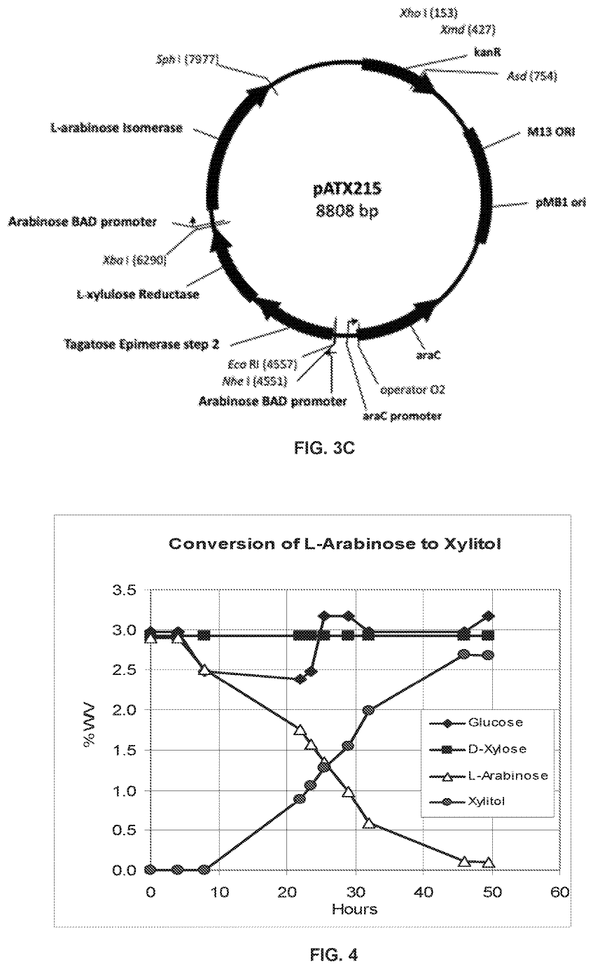

This plasmid was tested in E. coli ZUC134 (see U.S. 2006/0110809 incorporated herein by reference) for conversion of a synthetic hemicellulose mixture of D-xylose and L-arabinose to xylitol. Using this system, 27 g/L xylitol was produced from 50 g/L D-xylose without the production of any significant amount of arabitol. Higher concentrations of D-xylose did not result in more xylitol, and further study pinpointed the problem. Xylitol is inhibitory to XI activity, therefore a selection method was developed for creating xylitol resistant XI mutants. After several rounds of mutagenesis and selection, a more resistant XI was created and cloned into the expression vector to create pZUC052 (see U.S. 2006/110809 incorporated herein by reference). This mutant was capable of converting 150 g/L D-xylose to 74 g/L xylitol. However, with lower concentrations of D-xylose such as 30 g/L, conversion still was never more than 50% (FIG. 3). L-arabitol production from 30 g/L arabinose was insignificant.

### B. Convert L-arabinose to Xylitol, Reduce Xylose

Isomerize L-arabinose to L-ribulose; isomerize ribulose to L-xylulose; reduce L-xylulose to xylitol:

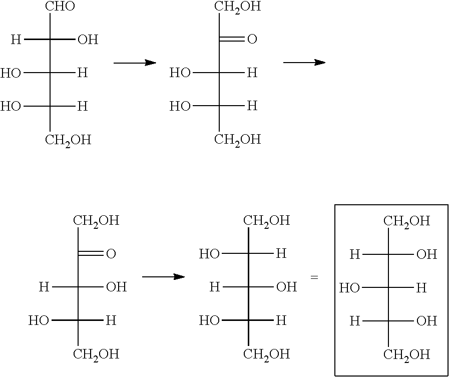 [SMILES: O=CC(O)C(O)C(O)CO ; O=C(CO)C(O)C(O)CO ; O=C(CO)C(O)C(O)CO ; OCC(O)C(O)C(O)CO ; OCC(O)C(O)C(O)CO]

### 1. Conversion of C-5 Mixed Sugars to Xylitol Via Epimerase Pathway.

FIG. 1C (Pathway C) depicts a pathway for converting L-arabinose to xylitol via an epimerase. Plasmid pATX210 (FIG. 3A) [U.S. patent application Ser. No. 11/827,506], Sakakibara et al. Methods for microbial production of xylitol from arabinose) contains an optimal combination of LAI from E. coli (araA) LXR from Ambrosiozyma monospora and DTE from Rhizobium radiobacter although alternative LAI, LXR and DTE genes could also be used. Plasmid pATX210 is a derivative of plasmid pBAD 18kan which contains an arabinose inducible promoter and kanamycin resistance marker. This plasmid was modified to contain a three gene cassette containing tagatose epimerase, L-xylulose reductase, and L-arabinose isomerase in that order moving away from the promoter. To test for the ability to convert a mixed sugar stream containing D-xylose, L-arabinose and other sugars, pATX210 was used to transform ZUC134, resulting in strain ZUC 136. As shown in FIG. 3B this strain has reproducibly been able to convert ˜90% of L-arabinose into xylitol (30 g/L to 27 g/L), while not consuming or modifying D-xylose (FIG. 4) in 48 hours.

### 2. Conversion of C-5 Sugar Mixtures to Xylitol—Two Stage Bioconversion (Path A and Path C Sequentially).

Another method of converting all of the xylose and arabinose to xylitol is to carry out a two-step sequential bioconversion using two different strains. For example, using strain ZUC136 (with the LAI/DTE/LXR pathway) to convert all of the L-arabinose to xylitol, optionally followed by a pasteurization or purification process to remove the original strain, followed by the use of ZUC140 (which contains the XR pathway) to convert the D-xylose in the resulting mixture to xylitol. If effective, the process will proceed without significant amounts of unwanted byproducts such as unreacted sugars or contaminating polyols being produced.

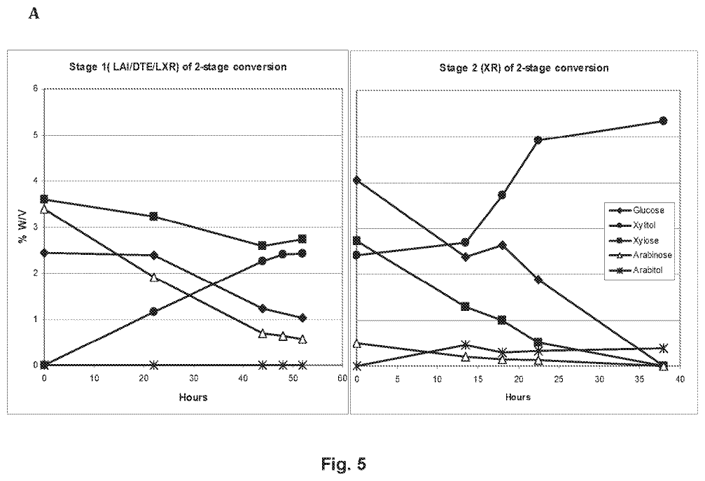

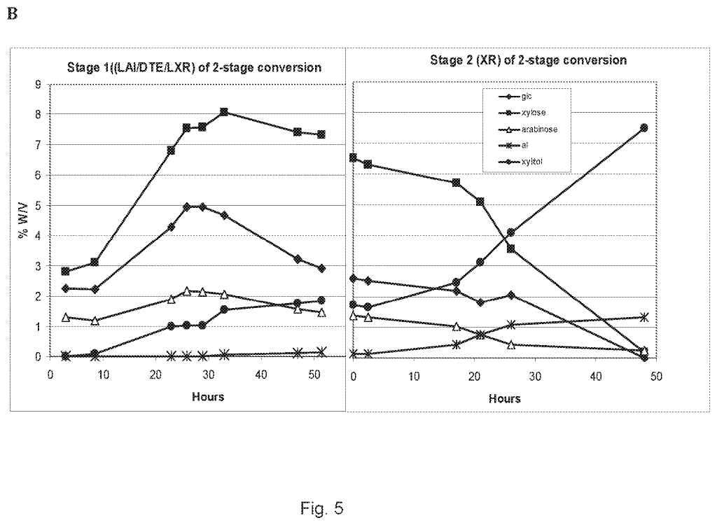

FIG. 5A shows the results of this strategy. The two-stage I L bioconversion started with a 50:50 synthetic hemicellulose (containing 33 g L-arabinose and 34.5 g D-xylose). The first stage bioconversion with ZUC136 lasted 50 hrs, and the second stage bioconversion with ZUC140 lasted 30 hrs. At the end of the bioconversion there was less than 8 g of combined other detectable sugars and polyols and the reaction produced approximately 65 g xylitol.

The process can also be run at higher concentrations of xylose and arabinose. As shown in FIG. 5B, Stage 1 proceeds until there are only small amounts of arabinose remaining unreacted. Stage 2 proceeds to completion converting all the xylose to xylitol. In this case a 2:1 synthetic hemicellulose feedstock was used with approximately 60 g D-xylose and 26 g L-arabinose. This process successfully produced 63 g xylitol at a concentration of 75 g/L.

During the two-stage bioconversion experiments, surprisingly the second stage, conversion of the xylose to xylitol was not only very rapid but did not generate a significant amount of arabitol even though there was some unreacted arabinose remaining in the broth. This was counter to expectations because most xylose reductase enzymes are known to convert both xylose to xylitol, and arabinose to arabitol. This was significant because the presence of excess amounts of arabitol in the final mixture would make final purification of xylitol overly expensive. Because of both the speed of the reaction, and the nature of the xylose reductase being used, the enzyme is more specific to xylose than other xylose reductases. The reaction proceeds without production of much arabitol when the reaction is slowed down, as it is in the second stage of the 2-stage conversion.

3. Conversion of C-5 Mixture to Xylitol Using a Single Strain with the Xylose Reductase and Epimerase Pathways Combined.

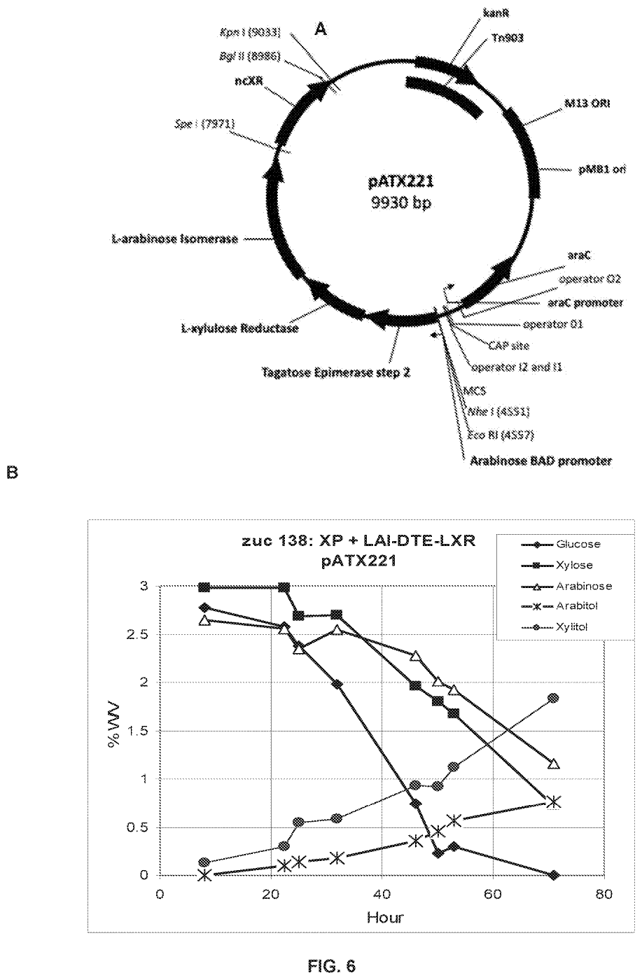

A way to convert both arabinose and xylose to xylitol is to put two separate pathways into a single organism. One combination of pathways in a single strain is the combination of Pathway A (XR) for converting D-xylose to xylitol, and Path C (LAI, DTE, LXR) for converting L-arabinose to xylitol. The primary issue is the production of L-arabitol from the activity of XR in the presence of L-arabinose. Combination of these pathways was achieved with the creation of pATX221 (created by insertion of the pTRP promoted ncXR into pATX2210 as depicted in FIG. 6(A)) which was subsequently transformed into ZUC134 to create ZUC138. The resulting strain grew and produced xylitol although slowly. In a 70 hr bioconversion this strain produced 20 g/L xylitol and 7 g/L arabitol from 30 g/L D-xylose and 26 g/L L-arabinose (FIG. 6(B)). To reduce the production of L-arabitol, a more D-xylose specific XR can be utilized.

4. Conversion of C-5 Mixture to Xylitol Using a Single Strain with the Xl/XDH and Epimerase Pathways Combined.

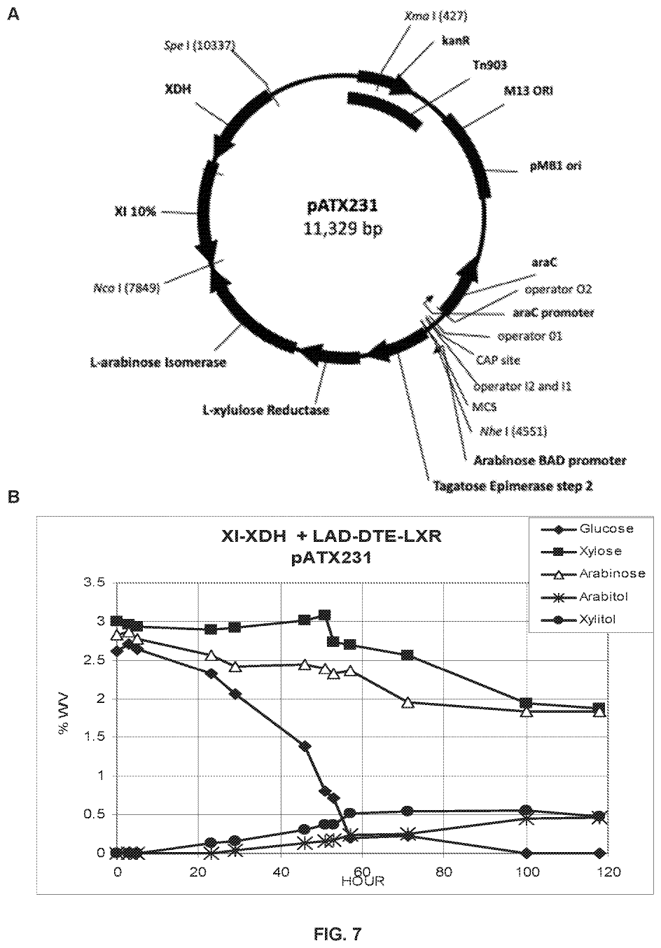

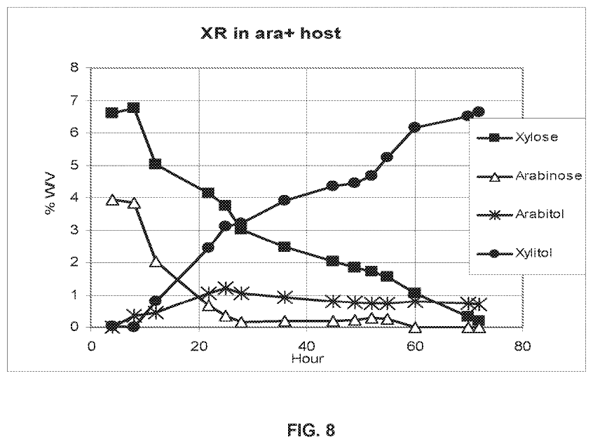

Another combination of pathways is to use Pathway B (XI, XDH) for D-xylose conversion and pathway C (LAI, DTE, LXR) for L-arabinose conversion. Plasmid pATX231 and pATX231 b were constructed with these combined pathways. These vectors were created by insertion of XDH and mutant XI into pATX210 as shown below in either the same orientation as the arabinose operon or in the reverse orientation. As seen in FIG. 7(A) the resulting recombinant strains produced xylitol although grew slowly and produced L-arabitol during bioconversion despite neither of the individual pathways producing L-arabitol on their own (FIG. 7(B)).

### C. Reduce D-xylose, Metabolize Arabinose

1. Conversion of C-5 Mixture to Xylitol Using Xylose Reductase in a Host that Metabolizes Arabinose.

Results of 2-stage bioconversion suggested the possibility that a system that produced xylitol with very little arabitol production could be generated by using a feedstock with a higher ratio of xylose:arabinose, although one that is still typical of many agricultural biomass products, and optimizing certain conditions. In this approach, the arabinose is metabolized as primary carbon source for the bioconversion.

To assess this method, the XR gene was placed in a host with wild type arabinose metabolism. E. coli strain ZUC 1 70 was created from E. coli B of the genotype F-ompT hsdSB(rB− mB−) gal dcm by transformation with the plasmid based vector pTRP-200 carrying NcXR and selection of the plasmid borne kanamycin resistance marker.

This strain was then tested with a synthetic hemicellulose containing a mixture of 6.8% xylose and 4% arabinose, a typical ratio for corn fiber hydrolysates. In a 72-hour bioconversion the yield of xylitol from xylose was excellent, more than 90%, and yielded 66 g/L, while less than 17.5% of arabinose was converted to arabitol at <7 g/L. Thus the final ratio of xylitol to arabitol was more than 8:1. Only a small amount of glucose was added, about 53 hours, which appeared to stimulate conversion. (FIG. 8)

A similar result was obtained using a strain, created in the same way as ZUC170, but with a more xylose specific xylose reductase created (VMQCI). With this strain (ZUC172) in the same xylose:arabinose mixture, more than 90% of xylose was converted to xylitol while 19.5% of arabinose was converted to arabitol at 6.9 g/L and a final ratio of more than 8:1.

This approach is especially attractive for hydrolysates with lower arabinose concentrations, such as many agricultural biomass sources (corn fiber, corn cob, etc.), woody biomass and any biomass that contains a xylose:arabinose ratio of approximately 3:1 or better. Using this route high concentrations of xylose from many of substrates are expected.

2. Production of Xylitol from a Hemicellulose Hydrolysate.

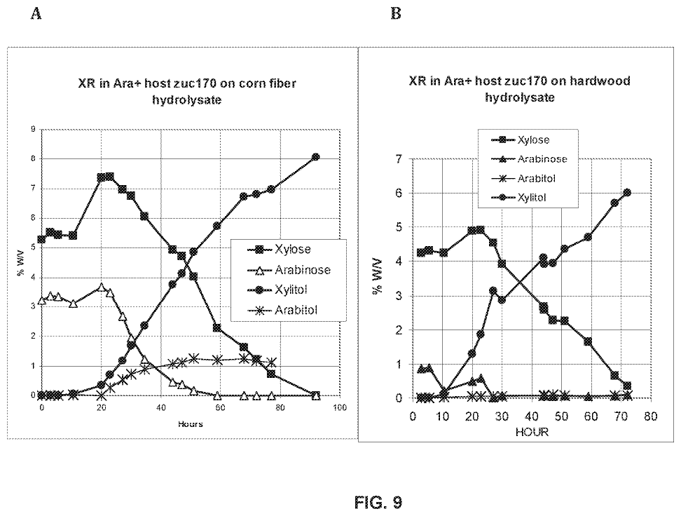

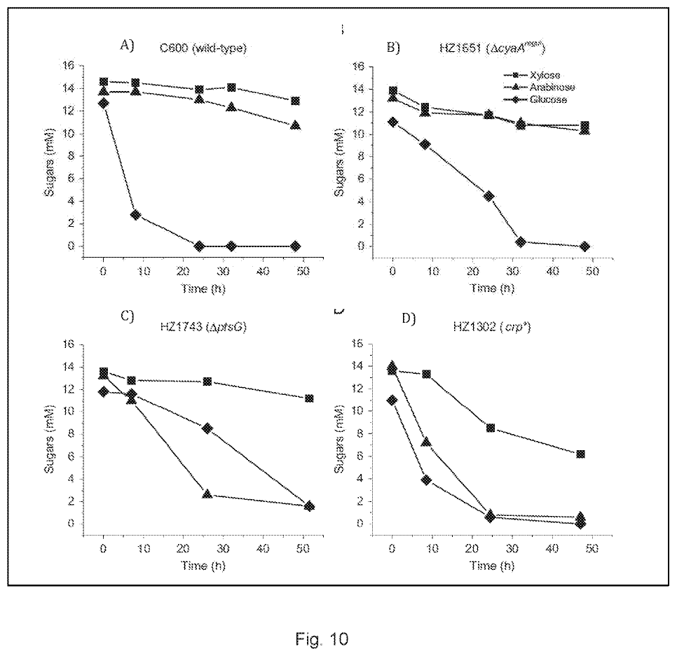

Production of xylitol in synthetic hemicellulose does not guarantee the process will work in a more complex and less pure biomass hydrolysate. To test utility of the system the ZUC170 strain fermented on different biomass hydrolysates. FIG. 9 shows results with a hydrolysate from corn fiber and woody fiber sources. Complete conversion was achieved in less than 80 hours to yield xylitol in concentrations between 60-80 g/L. In both cases the hydrolysates had been treated with overliming.

The corn fiber hydrolysate was fermented with a 1:1.5 dilution and grew and converted well. When arabinose was depleted, some glucose was added to maintain reducing power for xylose conversion. A final level of 80 g/L xylitol was achieved with near 100% conversion from xylose. (FIG. 9A)

Other hydrolysates are also suitable. For example, using the same volumes and organism, the hardwood hydrolysate that has a higher xylose to arabinose ratio (11.3% Xylose and 2.2% arabinose) can be used. In this case arabinose was consumed much sooner as there was less of it and thus less arabitol was formed (0.8 g/L vs. 60 g/L xylitol). This bioconversion finished in about 75 hours, and had a shorter lag. In this particular experiment, there was an over-addition of glucose at 44 hours which may have led to a slower bioconversion. Under these conditions very little arabitol was produced in both cases—even in the corn fiber hydrolysate which had significantly more arabinose to start with. (FIG. 9B).

Other hemicellulose hydrolysates such as those from corn fiber, corn stover, corn cob, bagasse, stillage, wheat straw, hardwood, softwood and other biomass sources are suitable.

### 3. Reduction of Lag Phase

One characteristic of these bioconversions is a lag phase of 12-15 hours at the beginning before xylitol production starts. Several approaches were tried to reduce this time. One approach was to use the broth from a well-grown fermenter at the peak of production, to inoculate a new fermenter.

Broth from fermenter 1 at 32 hours was used to inoculate fermenter 2 with the same medium composition. The second fermenter started producing xylitol without a lag and shows that with the proper inoculum, the bioconversion time can be reduced by about 12-15 hours. Another approach to increasing the rate, especially early in the bioconversion, would be to use a mutant that grows more rapidly in hydrolysate.

A nutrient solution consisting of 5 g tryptone 2.5 g yeast extract, and 1 g dipotassium phosphate was sterilized and added to a sterile fermenter. Corn fiber hydrolysate was detoxified by adding calcium hydroxide to pH 10.5, filtering over Whatman #1 paper, then neutralizing the filtrate with sulfuric acid and filtering again. A portion of this preparation, containing 13.2 g D-xylose, 4.8 g L-arabinose and 5.0 g D-glucose in 120 mL, was added without sterilization, before inoculation. The fermenter was inoculated with 25 ml of an overnight starter culture of ZUC170 grown in LB at 30° C. and run under the following conditions:

| | | |
|---|---|---|
| Temperature | 30° | C. |
| pH | 7.0 | (NH4OH control) |
| Air | 0.5 | LPM |
| Agitation | 800 | RPM |
| Volume after inoculation | 315 | ml |

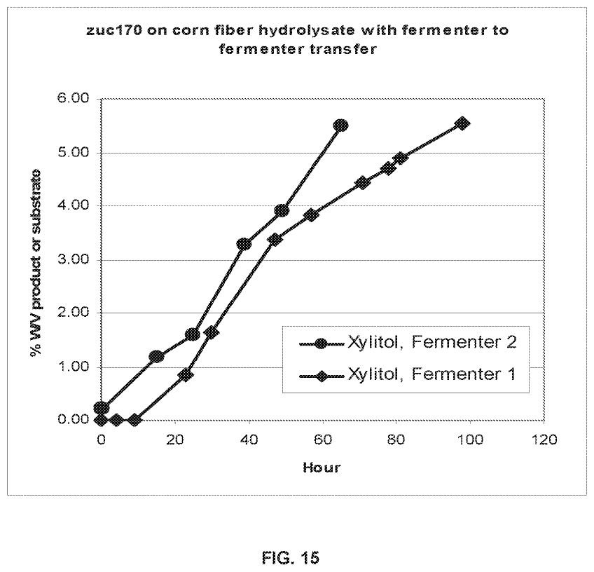

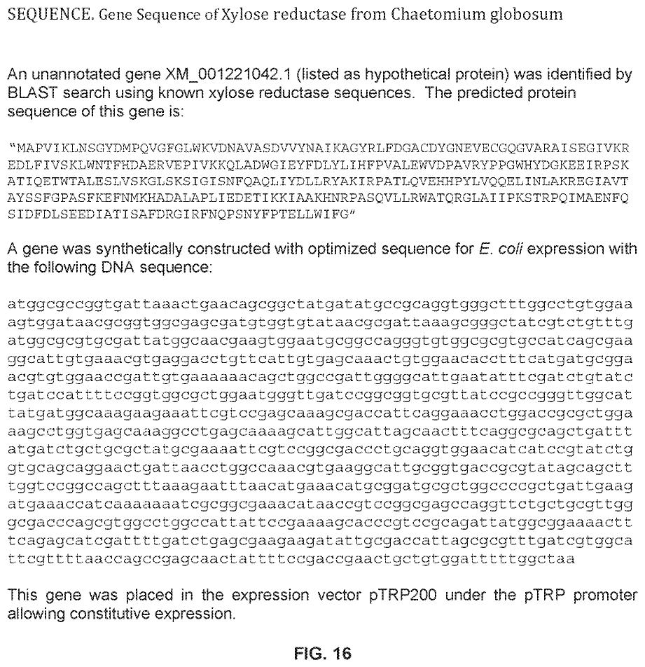

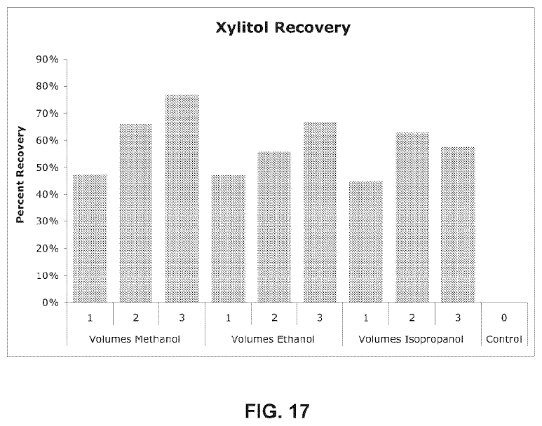

Additional detoxified hydrolysate containing 37.4 g D-xylose, 14.3 g L-arabinose and 13.6 g D-glucose in 340 mL was fed from 16-71 hours. Also, additional D-glucose, 124 g in 200 mL, was added from 24-98 hours. Growth and xylitol production initially lagged with no xylitol produced in the first 15 hours (FIG. 15, Fermenter 1). Then xylitol production began and continued until 46 g xylitol was produced in 98 hours in a final volume of 0.83 L. The volumetric productivity of xylitol was 0.56 g/L-h and the yield on glucose was 0.33 g/g.

To demonstrate that the productivity of the culture is not lost during the fermentation and to show the value of a larger inoculum adapted to growth in hydrolysate, a second fermentation (FIG. 15, Fermenter 2) was started using broth from this fermentation as inoculum. The inoculum for the second fermentation was 60 mL taken from the first fermenter 32 hours after inoculation. The second fermenter was run under the same conditions as the first. It produced the same amount of xylitol as first fermenter, but in 65 hours versus 98 hours. This was due to a reduced lag period and an increased rate.

### 4. Converting Xylose to Xylitol and Metabolizing; Reduce D-Xylose by Novel Microorganisms to Produce Xylitol

A xylose reductase (XR) was previously isolated from the filamentous fungus Neurospora crassa. The enzyme has an innate 2.4-fold preference for D-xylose over L-arabinose. Resting cell studies in recombinant E. coli expressing this enzyme demonstrated that such a small difference in selectivity was sufficient to improve the ratio of xylitol-to-arabinitol produced. To increase the selectivity of the process toward xylitol, the XR for decreased L-arabinose reductase activity was engineered, and via several rounds of directed evolution, a mutant designated VMQCI was isolated that had a 50-fold lower catalytic efficiency toward L-arabinose. This mutant retained <2% of its original L-arabinose reductase activity. Resting cell studies with this mutant revealed that although the amount of L-arabinitol was significantly decreased, it was not completely eliminated. In order to further increase the selectivity of this biocatalytic process, an orthogonal strategy was implemented to reduce final L-arabinitol titer. For this purpose a metabolically engineered E. coli strain was created that is highly efficient at utilizing L-arabinose as a carbon source, and able to sequester it away from XR, decreasing L-arabinitol production.

By combining the engineered protein with a metabolic engineering strategy
a combination that is contemplated creates biocatalysts with novel properties and synergism.

Xylitol can be made from a better than 1:1 ratio of xylose to arabinose. Fermenting microorganisms were sought to facilitate xylitol production. Of particular concern is the need to reduce arabinitol to a negligible amount, or to convert arabinose to xylitol. Some microorganisms have been reported to achieve these goals but have limitations. One of the major obstacles to creating a strain that is highly efficient at utilizing L-arabinose as a carbon source, is that the regulation of various catabolic pathways of E. coli in the presence of multiple sugars is not well understood. This is particularly important for selective production of xylitol from hemicellulosic hydrolysate since corn fiber consists of D-xylose, L-arabinose, and D-glucose. While diauxic growth patterns due to glucose repression in E. coli is well studied, little is known about the relative preference between pentoses, and even less in the presence of glucose. In addition, a system used to overexpress XR is IPTG (isopropyl-β-D-thiogalactopyranoside)-dependent, which is reliant on the lactose system, introducing a fourth regulatory system. Considering that the transport of all three non-glucose sugars is dependent on CRP (cyclic adenosine monophosphate receptor protein), significant cross-talk between them is to be expected. Glucose de-repression for simultaneous uptake of two sugars has been documented previously, albeit primarily for ethanol production, which was carried out under oxygen-limited conditions. The pleiotropic effects on other regulatory systems of such de-repressed mutants are poorly characterized.

To engineer E. coli for efficient L-arabinose catabolism in the presence of glucose and D-xylose, three different de-repression strategies were used: a glucose phosphotransferase mutant, a regulation deficient adenylate cyclase mutant, and a CRP mutant (Goerke and Stulke, 2008). The crp* mutant can be superior among the three under certain conditions. This mutant was previously described to be helpful in co-utilization of D-xylose and glucose for the production of xylitol using an IPTG induction system. In this strain, the effects of overexpressing a xylose transporter (XylE) were tested as well as the relative productivity of placing XR under the control of D-xylose-, IPTG-, and L-arabinose-inducible systems. Under certain conditions, L-arabinose was preferred over glucose, whereas under other growth conditions glucose was the preferred carbon source. Finally, in a bioreactor setting, the engineered strain in conjunction with the mutant XR (VMQCI) was able to eliminate L-arabinitol production from an equiweight mixture of D-xylose, L-arabinose and glucose.

### Under Some Conditions Using the 1:1 Mixture of Arabinose:Xylose the crp * Mutant is the Most Efficient at Co-Utilizing Three Sugars for Xylitol Production.

Three different catabolite de-repression strategies HZ1743, HZ1651 and HZ1302 (ΔptsG, ΔcyaAreg, and crp*, respectively were tested for co-utilization of glucose, D-xylose and L-arabinose. The phosphotransferase system (PTS) for simultaneous glucose uptake and phosphorylation has been shown to play a role in catabolite repression (Goerke and Stulke, 2008). Strains with inactivated permease, PtsG, were shown to relieve the repression and have been used for co-fermenting mixed sugars (Nichols et al., 2001). Adenylate cyclase (CyaA) is responsible for forming cAMP in response to low glucose concentrations. Its activity is regulated by interaction with the PTS protein Enzyme IIAGlc. A strain with truncated CyaA was shown to be de-regulated and did not demonstrate diauxic behavior when grown in glucose and maltose mixtures (Crasnier et al., 1994). Several CRP (also known as CAP, catabolite activator protein) mutants have been isolated that show de-repressed behavior (Eppler and Boos, 1999; Karimova et al., 2004; Zhu and Lin, 1988). For the present disclosure, the CRP mutant that was shown to de-repress xylose metabolism under aerobic conditions for xylitol production, was used (Cirino et al., 2006; Eppler and Boos, 1999).

Deletions were created by replacing the undesired locus with PCR amplified cat (CmR) mediated by λ red recombinase proteins (Datsenko and Wanner, 2000), either directly in the parent strain, or in MG 1655 and then transduced into the appropriate recipient Miller, 1992). The CRP mutant was created by transduction of donor allele from ET23 into C600 (Eppler and Boos, 1999; Miller, 1992).

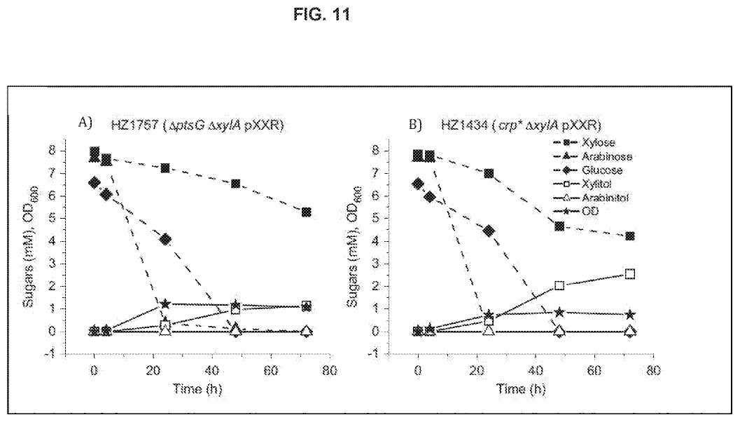

These three recombinant strains plus the wild type strain were grown in minimal medium with ˜2 g/L each of glucose, D-xylose and L-arabinose under oxygen-limited conditions. Supernatants were analyzed at various time points to ascertain their sugar utilization patterns (FIG. 10). The wild-type C600 (FIG. 10A) demonstrated strong diauxie, with almost no uptake of D-xylose or L-arabinose until complete depletion of glucose. The strain with truncated CyaA (HZ1651) (FIG. 10B) showed slightly decreased glucose assimilation, although pentose utilization was not significantly improved. The PtsG knockout (HZ1743) (FIG. 10C) demonstrated delayed response to glucose, but was able to uptake L-arabinose and glucose simultaneously, albeit with differing rates. Finally, the crp* mutant (HZ1302) (FIG. 10D) showed efficient simultaneous assimilation of all three sugars, although, as in all strains, xylose uptake was the slowest. Based on these data, HZ1651 was deemed unsuitable for xylitol production. After deletion of XylA in HZ1743 and HZ1302 to prevent xylose catabolism, pXXR (wtXR under XylA promoter) was transformed into both strains to give HZ1757 (FIG. 11A) and HZ1434 (FIG. 11B), respectively, and tested for xylitol productivity. Although both strains demonstrated efficient utilization of glucose and L-arabinose as carbon sources, the stronger induction from xylose promoters in HZ1434 is evident from higher xylose conversion to xylitol. Based on these experiments, the crp* mutant strain was used for further engineering work

Crabtree Effect is Prevalent at High Sugar Concentrations in the crp* Strain

Glycolysis rate at high sugar concentrations often exceeds respiratory capacity, leading to build-up of intermediate metabolites. This “Crabtree effect” is well-known for many organisms including S. cerevisiae and E. coli, which are known to build up ethanol and acetate, respectively. In E. coli acetate build-up decreases growth rate as well as recombinant protein production. Previous work in a similar crp* strain showed that at 18 g/L glucose concentration, acetate production is significant, accumulating to 70 mM.

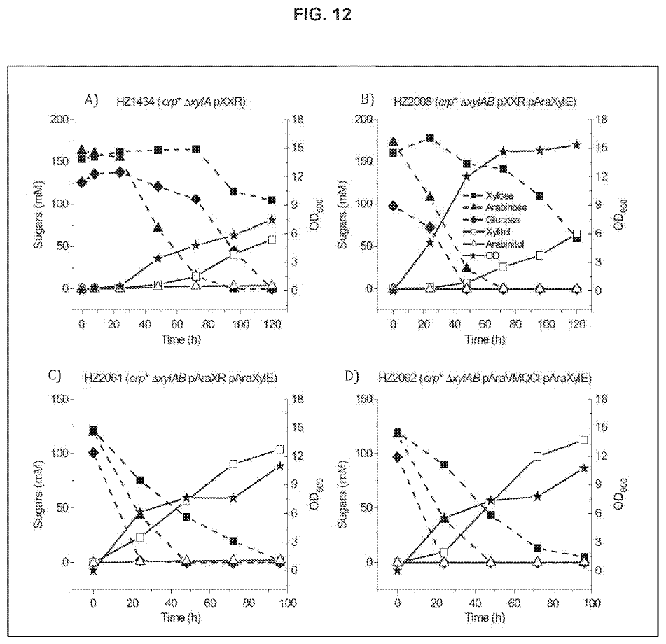

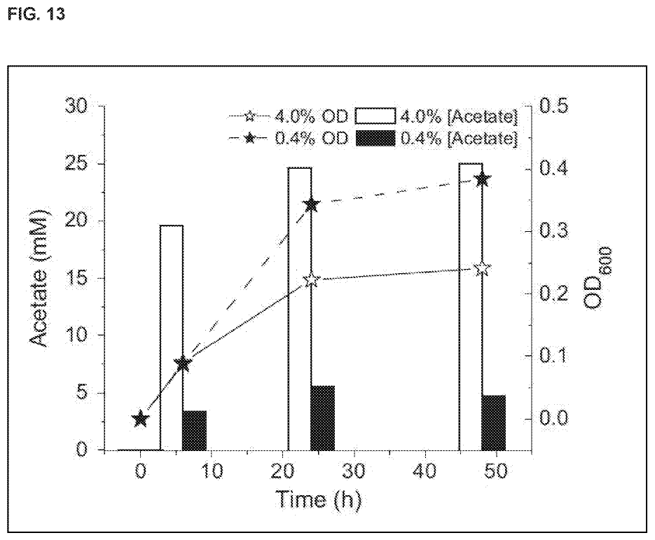

When HZ1434 was grown in 40 g/L total usable sugar (glucose+L-arabinose) in minimal M9 medium, pH dropped to ˜5 within 24 hours, completely inhibiting growth due to high level acetate production (FIG. 12). Addition of 50 mM MOPS (4-morpholinopropanesulfonic acid) to the medium could not buffer the pH at 7.0, as had been done previously at 18 g/L glucose. Addition of a complex nitrogen source has been shown to reduce acetate production in batch cultures (Panda et al., 2000). However, addition of 10 g/L tryptone did not prevent acid accumulation. Although genetic methods exist to decrease acetate production pleiotropic effects could lead to additional complications. Therefore, a pH-stat bioreactor was used subsequently.

Expression from Arabinose Promoter Decreases Crabtree Effect and Lag Phase

In the pH-stat bioreactor with 60 g/L total sugars (equiweight D-xylose, L-arabinose, and glucose), there was a 24 h lag phase. In addition, xylitol production was minimal until near-complete depletion of L-arabinose in the medium (FIG. 12A). Poor induction of the xylose pathway compared to the arabinose operon (FIG. 10D) was likely the primary reason for low productivity. Since overexpression of xylose-proton symporter (XylE) was shown to transport D-xylose efficiently in glucose-xylose mixtures (Khankal et al., 2008), it may help increase xylitol productivity. Expression using a constitutive promoter, BLMAp (Kim et al., 2003) using pACYCBLMAXylE in HZ2009 (Table 1), did not improve xylitol conversion (data not shown). On the other hand, expression of XylE under the AraBAD promoter from a multicopy plasmid (pACYCAraXylE) (SEQ ID NO: 21) had the unexpected side-effect of simultaneously decreasing both the lag phase of HZ2008 and the total amount of alkali required to maintain pH at 7.0 (FIG. 12B). Unfortunately, the xylitol productivity was nearly unaltered. Another side-effect of this is the change of the relative rates of glucose and L-arabinose consumption. Prior to XylE overexpression (HZ1434), L-arabinose was assimilated faster than glucose (FIG. 11B, 12A), whereas after its overexpression (HZ2008), glucose was the preferred carbon source (FIG. 12B). It is a possible that promoter dilution may play a role in decreasing expression from the chromosomal araBAD operon, although previous reports indicate that this phenomenon is not significant in bacteria. Alternately, the presence of XylE in the cell membrane either replacing AraE and AraGFH transporters, or in addition to them, could be retarding the rate of L-arabinose uptake. This could also explain the lower requirement for alkali in the bioreactor, since the respiration rate would be more capable of keeping up with the slower glycolysis of L-arabinose.

since overexpression of XylE did not improve the final xylitol titer, the poor productivity was likely due to low expression of XR under the control of XylA promoter, despite its extremely high activity. So, XR was placed under either the IPTG-inducible Trc promoter (pTrcXR) or the AraBAD promoter (pAraXR). Induction from a lac-based promoter in crp* strain in glucose-xylose mixtures was previously shown to produce high levels of recombinant protein, even at 100 μM concentration (Cirino et al., 2006). However, expression of XR from the Trc promoter induced with 100 μM IPTG led to even poorer conversion than that obtained using the XylA promoter (HZ2046, data not shown). Under the AraBAD promoter (HZ2061), xylitol production reached near stoichiometric levels, with low levels of L-arabinitol production as well (2-6 mM, FIG. 12C). The VMQCI mutant produced xylitol at a slightly slower rate than wtXR (HZ2062), as would be expected from the lower overall activity of the mutant (FIG. 12D), but it produced undetectable levels of L-arabinitol over the 4 day period (limit of detection <1 mM).

### Catabolic Pathways: Activation and Competition

Catabolic pathways for sugars other than glucose are normally repressed in its presence. Four different strategies for de-repression were tested and the crp* mutant was the most efficient at simultaneously activating the D-xylose and L-arabinose metabolic pathways (FIG. 10). However, the arabinose pathway was more strongly activated, as evident from quicker uptake and assimilation compared to D-xylose. Using XR as a reporter under the control of arabinose (AraBAD), xylose (XylA), or lactose (Trc) promoter systems, AraBAD was the most strongly expressed among all three. Although the lac-based system was shown to be fully activatable with 100 μM IPTG in crp* strains in the presence of glucose and D-xylose (Cirino et al, 2006), in the presence of three sugars, this promoter was weakly induced. This is true even in light of the fact that IPTG is the only non-transformable inducer tested. In a non-crp* strain, there is strong activation of D-xylose, L-arabinose, and lactose operons simultaneously in the absence of glucose. Lee and coworkers (2007) have shown that presence of IPTG represses AraBAD promoter activation.

In contrast to these observations, in the crp* strain created here, the exact opposite was found—AraBAD repressed activation from IPTG-dependent promoters. Investigations into the mechanism of competition and cross-talk between the regulation of three non-glucose operons in wild-type and crp* strains in the presence or absence of glucose would help explain the behavior seen here. The roles of sugar-specific transporters and transcription activators/repressors, in particular, would reveal the mechanism of these interactions. The combination of protein engineering and metabolic engineering led to synergistic increase in desired biocatalytic properties. In this particular case, the synergy was manifested as increased selectivity such that that L-arabinitol production was minimal.

To realize this goal, a metabolically engineered E. coli strain was created that is highly efficient at utilizing L-arabinose as a carbon source, and able to sequester it away from XR, decreasing L-arabinitol production. One of the major obstacles to create such a strain was that the regulation of various catabolic pathways of E. coli in the presence of multiple sugars is not well understood. This is particularly important for selective production of xylitol from hemicellulosic hydrolysate because corn fiber consists of D-xylose, L-arabinose, and D-glucose. Although diauxic growth pattern due to glucose repression in E. coli is well studied, little is known about the relative preference between pentoses, and even less in the presence of glucose. In addition, a system described herein to overexpress XR is IPTG (isopropyl-β-D-thiogalactopyranoside)-dependent, which is reliant on the lactose system, thus introducing a fourth regulatory system. Considering that the metabolism of all three non-glucose sugars is dependent on activation by CRP (cyclic adenosine monophosphate receptor protein), significant cross-talk between them is to be expected. Glucose de-repression for simultaneous uptake of two sugars has been documented previously, albeit primarily for ethanol production, which was carried out under oxygen-limited conditions (Lindsay et al., 1995; Nichols et al., 2001). The pleiotropic effects on other regulatory systems of such de-repressed mutants are poorly characterized.

L-arabinitol production can be almost completely eliminated from an equiweight mixture of D-xylose, L-arabinose, and glucose—the three major sugars in hemicellulosic hydrolysate. Considering actual corn hemicellulose has D-xylose to L-arabinose in a 5:3 ratio, the tested equiweight mixture is a worst-case scenario. This strategy used an engineered E. coli strain with glucose depressed growth and xylose transporter overexpression to quickly assimilate L-arabinose as a carbon source, sequestering it away from the substrate selective XR mutant VMQCI. Not only is L-arabinose prevented from being converted to L-arabinitol, it also provides reducing equivalents in the form of NADPH for xylitol production, and acts as an inducer for protein expression.

### 5. Improved Strain (ZU220) for Conversion of Hemicellulose to Xylitol

A new strain with significant improvement in yield of xylitol per gram of glucose and per gram of base was developed and named ZUC220. ZUC220 (xylBΔ, ptsGΔ-glucose selected pTRP200-ncXR) was created by PCR-based genetic deletion of xylB and ptsG from starting strain AB707 (K12 prototroph), followed by selection on glucose containing minimal medium for several generations, and then the resulting strain was transformed with pTRP200-ncXR (constitutive expression vector containing ncXR).

The volumetric productivity of ZUC220 is higher than ZUC170.

### Use of ZUC220 on Synthetic Mixture of Sugars

| | | |
|---|---|---|
| Tryptone | 14 | g |
| Yeast extract | 7 | g |
| Potassium phosphate, | 4.2 | g |
| dibasic | | |
| Sodium chloride | 7 | g |
| Magnsesium sulfate | 2 | g |
| Water | 750 | mL |
| Antifoam Cognis Clerol | 3 | drops |
| FBA 3107 | | |

The vessels were sterilized with the above media in situ. D-xylose (30 g) and D-glucose (30 g) was sterilized in 100 ml water separately and added prior to inoculation of the vessel. The fermenters were inoculated with 50 ml of an overnight starter culture grown in LB at 30° C. and run under the following conditions:

| | | |
|---|---|---|
| Temperature | 30° | C. |
| pH | 7.0 | (NH4OH control) |
| Air | 1 | LPM (1 VVM) |
| Agitation | 800 | RPM |
| Volume after inoculation | 900 | ml |

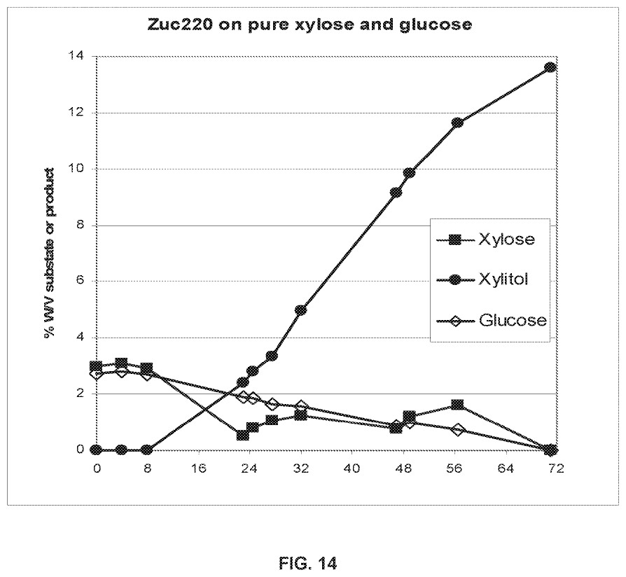

A feed of D-xylose (130 g) and D-glucose (40 g) was dissolved in 185 ml water, sterilized and used to feed the fermentation from 23-56 hours after inoculation. The result was 156 g xylitol produced in 71 hours in a final volume of 1.145 L(136 g/L concentration (FIG. 14A). The volumetric productivity was 1.92 g/L-h, nearly twice the rate previously obtained with ZUC170 (FIG. 8). The yields on glucose and base were 2.48 g xylitol per g glucose and 46 g xylitol per g NH₄OH.

The medium was sterilized and added to a sterile fermenter. Corn fiber hydrolysate was detoxified by adding calcium hydroxide to pH 10.5, filtering over Whatman #1 paper, then neutralizing the filtrate with sulfuric acid and filtering again. A portion of this preparation, containing 13.2 g D-xylose, 4.8 g L-arabinose and 5.0 g D-glucose in 120 mL, was added without sterilization, before inoculation. The fermenter was inoculated with 25 ml of an overnight starter culture of ZUC170 grown in LB at 30° C. and run under the following conditions:

| | | |
|---|---|---|
| Temperature | 30° | C. |
| pH | 7.0 | (NH4OH control) |
| Air | 0.5 | LPM |
| Agitation | 800 | RPM |
| Volume after inoculation | 315 | ml |

Additional detoxified hydrolysate containing 37.4 g D-xylose, 14.3 g L-arabinose and 13.6 g D-glucose in 340 mL was fed from 16-71 hours. Also, additional D-glucose, 124 g in 200 mL, was added from 24-98 hours. Growth and xylitol production initially lagged with no xylitol produced in the first 15 hours (FIG. 14B, Fermenter 1). Then xylitol production began and continued until 46 g xylitol was produced in 98 hours in a final volume of 0.83 L. The volumetric productivity of xylitol was 0.56 g/L-h and the yield on glucose was 0.33 g/g.

To demonstrate that the productivity of the culture is not lost during the fermentation and to show the value of a larger inoculum adapted to growth in hydrolysate, a second fermentation (FIG. 14B) was started using broth from this fermentation as inoculum. The inoculum for the second fermentation was 60 mL taken from the first fermenter 32 hours after inoculation. The second fermenter was run under the same conditions as the first. It produced the same amount of xylitol as first fermenter, but in 65 hours versus 98 hours. This was due to a reduced lag period and an increased rate.

### II. Crystallization

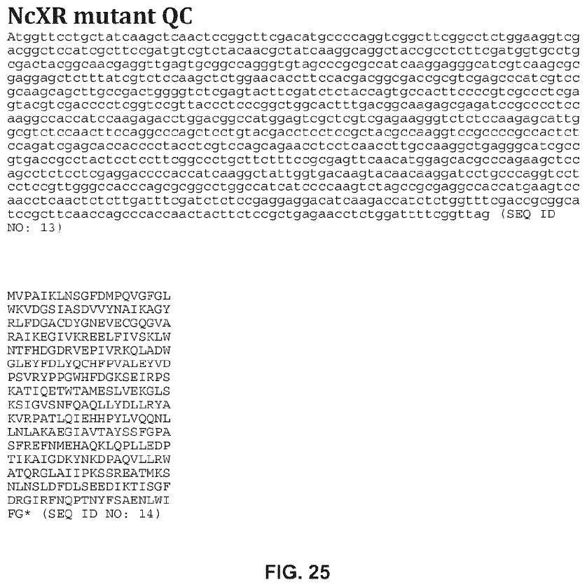

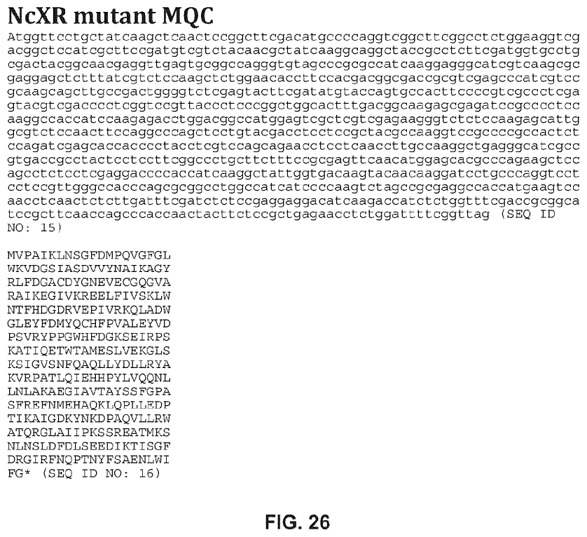

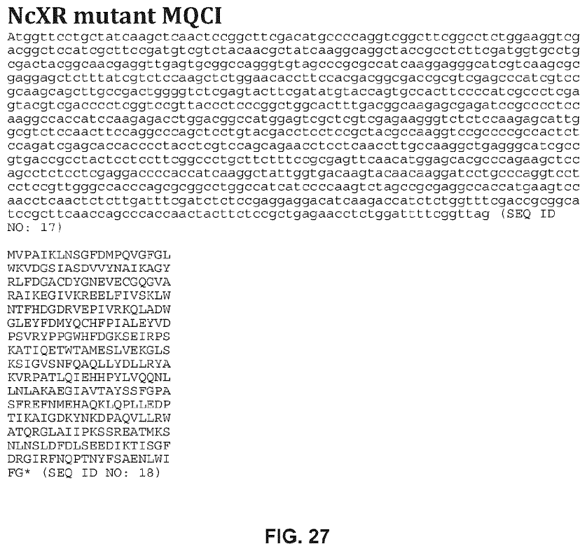

A. Xylitol with Cosolvents. In order to test the effect of co-solvents on crystallization of xylitol, a 50% solution of xylitol was separated into 10 mL aliquots and various quantities of cosolvents (methanol, ethanol, and isopropanol) were added. The mixtures were allowed to crystallize overnight at −20° C. and inspected. Only a small (<10%) amount of crystallization was noted. A separate experiment was carried out using the same methodology, but with seeding using 1 mg of finely ground xylitol crystals. After overnight crystallization, significant xylitol crystallization was obtained. These crystals were removed by filtration, washed with a small amount of cosolvent, dried, and the mass was recorded. The various recoveries are displayed in FIG. 25. The best recovery was approximately 80% of the initial xylitol in solution in a single stage of crystallization using 3 volumes of methanol. A control containing no cosolvent did not result in any xylitol formation. These initial conditions are very promising and should afford the desired yield of recovery.

B. Methods. Crystallization from bioconversion broths can be achieved in a number of ways. One way is to subject the bioconversion broth to charcoal treatment, followed by concentration of the xylitol-containing broth to a xylitol concentration of around 700 g/L. Treatment of concentrated bioconversion broth with cation exchange calcium affinity chromatography helps speed the crystallization. To date a single simple chromatography step helps remove salts and other byproducts and improves crystallization. As high as 80% recovery was achieved with the final material meeting the desired purity specifications. Recovery can include some or all of the following steps:
Cell removal. Microfiltration, centrifugation, or vacuum filtration is required (rotary drum filter).
Charcoal treatment. The cell-free broth is mixed with 5 g/L activated charcoal. Mixing is continued for 1 hour at 37° C., and then the charcoal is separated by filtration on a filter press. Alternatively, a charcoal column can be used.
Evaporation. The volume is reduced by removing 80% of the volume by evaporation under vacuum at 55-60° C. Target, 500-700 g/L xylitol. An efficient multistage evaporator is required.
Cation exchange. To remove salts and other byproducts.
Crystallization. The concentrate is cooled to induce crystallization. A crystallizer is required. Crystallization may be induced by addition of seed crystals or alcohol cosolvent such as methanol, ethanol, or isopropanol.
Crystal collection and washing. A basket centrifuge or Nutsche filter is required. The crystals are collected and washed free of impurities.
Drying. A fluid bed dryer can be used.
Recrystallization. If needed, the xylitol can be further purified by undergoing a recrystallization process.

### Supplemental Materials and Methods

### Materials

All media were purchased from Becton-Dickinson (BD, Sparks, MD), chemicals from Sigma-Aldrich (St. Louis, MO), enzymes from New England Biolabs (NEB, Beverly, MA), and oligonucleotide primers from Integrated DNA Technologies (IDT, Coralville, IA). All DNA purification kits were obtained from Qiagen (Valencia, CA), except that the Wizard® Genomic DNA Purification Kit was procured from Promega (Madison, WI). Cells were maintained on Lysogeny Broth (LB) plates containing 1.5% agar and the appropriate antibiotic. Selection for plasmid maintenance was done with ampicillin (100 mg/L), chloramphenicol (25 mg/L), and kanamycin (50 mg/L). Chromosomal integrants were selected on chloramphenicol (6 mg/L) or tetracycline (10 mg/L) LB plates.

### Plasmid Construction

All cloning work was performed in E. coli DH5α or WM1788 (pir+ for propagating R6K plasmids), and a list of constructs can be found in Table 1. All XR expression plasmids were derivates of pTrc99A. XR and mutants were previously cloned into pACYCDuet (Novagen), and were used as the template for PCR (Nair and Zhao, 2008). The XylA promoter was amplified from E. coli MG 1655 genomic DNA, and spliced with XR using overlap extension PCR. The cassette was digested with NsiI and BglII and ligated into pTrc99A that had been digested with NsiI and BamHI. Ligation of compatible BglII-BamHI ends abolished both restriction sites. The AraBAD promoter was digested out of pRW2-ptdh (Johannes et al., 2005) using Pstl and NdeI; PCR amplified XR was digested with NdeI and BglII, and pTrc99A with NsiI and BamHI. All three were ligated together in a single reaction, which abolished the compatible PstI-NsiI and BglII-BamHI sites. For IPTG inducible constructs, XR (EcoRI-BglII) was directly ligated into EcoRI-BamHI digested pTrc99A. Xylose transporter xylE was amplified from MG1655 genomic DNA and ligated directly into pTKXb-xdh-araB′ (Kim et al., 2003; Nair and Zhao, 2008) digested with NdeI and XhoI. The promoter-gene cassette was then digested out with EcoRI and XhoI and ligated in pACYCDuet digested with the same endonucleases. This construct provided expression from the constitutive BLMA promoter. For expression under the AraBAD promoter, xylE was first cloned into pRW2-ptdh between the NdeI and PciI sites. The promoter-gene cassette was then digested out using PstI and PciI and ligated into pACYCDuet digested with PstI and NcoI. The ligation abolished the compatible NcoI-PciI sites.

### Genetic Methods

All strains used for xylitol production were E. coli K-12 C600 and its derivates (Table 1), and all deletions were performed using the y red system (Datsenko and Wanner, 2000). Briefly, PCR product containing the cat gene flanked by FRT (Flp recognition target) and 45-50 nt of sequence identical to the target locus was transformed into cells expressing y red recombinase proteins (encoded on pKD46). Gene replacement was selected on chloramphenicol plates and verified by functional assay and PCR. The resistance marker was then removed by the expression of Flp recombinases from a thermo-inducible promoter on a temperature sensitive plasmid (pCP20). Flp recombinase plasmid loss and cat loss occurred simultaneously and were verified by sensitivity to ampicillin and chloramphenicol. Deletion of ptsG and cyaA^{regul} was performed directly in C600, whereas inactivation of the xylA and xylAB genes was performed in MG 1655 and then moved by P1 transduction to the recipient strains (Miller, 1992). The crp* mutation was also generated by P1 transduction from ET23 and selecting for Tet^{R} integrants (Eppler and Boos, 1999). Deletions were verified by PCR using cell lysate as the template and appropriate flanking primers. Verification of glucose de-repression was first done by blue/white screening on LB plates containing 10 g/L glucose. Strong induction of lacZ in the presence of glucose indicated the depressed phenotype. The CyaA mutant strain did not demonstrate significant LacZ activity. Finally, direct monitoring of sugar co-utilization in shake flasks was used to verify de-repression.

### HPLC Analysis

Sugar concentrations were quantified using Shimadzu high performance liquid chromatography (HPLC) equipped with a low temperature evaporative light scattering detector (ELSD-LT) (Columbia, MD). A Bio-Rad Aminex 250×4 mm HPX-87C (Bio-Rad, Hercules, CA) carbohydrate column was used to separate the sugars, as per manufacturer's recommendations. The column was run at 0.2 mL/min at 85° C. for 18 minutes with water as the mobile phase.

### GC-MS Analysis

Acetate quantification was performed at the Roy J. Carver Metabolomics Center. n-Butanol (1 mL/L) was used as internal standard to quantify acetate in media. Samples (1 μl) were injected in split mode (5:1) to the GC/MS system consisting of an Agilent 7890 gas chromatography, an Agilent 5975 mass selective detector, and HP 76838 autosampler (Agilent Technologies, Palo Alto, CA). Acetate samples were analyzed on a 30 m ZB-Wax-Plus column with 0.32 mm I.D. and 0.25 μm film thickness Phenomenex, Torrance, CA) with an injection port temperature of 250° C., the interface set to 250° C., and the ion source adjusted to 230° C. The helium carrier gas was set at a constant flow rate of 2.5 mL min−1. The temperature program was 5 min isothermal heating at 90° C., followed by an oven temperature increase of 10° C. min-1 to 210° C. for 2 min. The mass spectrometer was operated in positive electron impact mode (EI) at 69.9 eV ionization energy in m/z 50-550 scan range.

The spectra of all chromatogram peak was evaluated using the HP Chemstation program (Agilent Technologies, Palo Alto, CA). Identification was performed using the mass spectra obtained from the authentic standards and additionally confirmed with NISTO8 and W8N08 libraries.

### Shake Flask and Bioreactor Cultures

For shake flask cultures, overnight cultures were grown at 37° C. in M9 minimal medium supplemented with 2 mM MgSO4, 0.1 mM CaCl₂), 20 mg/L leucine, 120 mg/L threonine, 10 mg/L thiamine-HCl, 2 g/L glucose and the appropriate antibiotic(s). 125 mL unbaffled bottles containing 25 mL of the same medium but containing 1-2 g/L of each sugar (glucose, D-xylose, and L-arabinose) were placed under vacuum, filled with nitrogen, and capped with airtight stoppers to maintain oxygen-limited conditions. 1 mL overnight cultures were inoculated into these bottles and maintained at 30° C. or 37° C. at 250 rpm. For bioreactor studies, 4 mL overnight cultures were grown at 37° C. either in LB or M9 medium supplemented with 2 mM MgSO4, 0.1 mM CaCl₂), 20 g/L glucose, 10 g/L tryptone, and the appropriate antibiotic(s). Upon reaching saturation, these cultures were spun down and resuspended in 4 mL of the same medium and cultured for another 4 hours. These cultures were then inoculated into 400 mL bioreactors containing the same M9+tryptone medium with additional 20 g/L each of D-xylose and L-arabinose, as well as antifoam agents. Bioreactors were run at 30° C. with 400 rpm agitation and 0.8 L/min sparging with air. pH was maintained at 7.0±0.1 with 5N NaOH and 2 NH₂SO₄.

Patrick C. Cirino (Pennsylvania State University, PA) provided the crp* parent strain ET23, William W. Metcalf (UIUC) provided the pir+ cloning strain WM1788, and John E. Cronan (UIUC) provided P1 vir phage used for transduction.
(a) sequential fermentation of both arabinose and xylose to xylitol—using two microbial strains. In this process a high arabinose:xylose concentration (>1:1) may be used;
(b) parallel fermentation of both arabinose and xylose to xylitol using a single microbial strain. Two different systems were developed;
(c) conversion of xylose to xylitol with consumption of arabinose using a moderate arabinose:xylitol ratio (>1:3) without a mutation designated CRP. Productivity is about 10× the CRP system. Examples also support that this is an unexpected result. A fermentation system that converts a mixed C5 sugar stream to low-arabitol product uses a CRP (cyclic adenosine monophosphate receptor protein) mutation useful with both the wild-type and mutant XR; and
(d) demonstration of a fermentation system using both synthetic hemicellulose and a variety of industrial hemicellulose samples.

| ABBREV | Enzyme Name | Function |
|---|---|---|
| XR (AR) | Xylose (or Aldose) | Converts xylose |
| | Reductase | (and arabinose) |
| | | to xylitol |
| | | (and arabitol) |
| XI | Xylose | Isornerizes xylose |
| | isomerase | into d-xylulose |
| XDH | Xylitol | Converts between |
| | Dehydrogenase | d-xylulose |
| | | and xylitol |
| LXR | l-xylulose | Converts l-xylulose |
| | reductase | to xylitol |
| LAI | l-arabinose | Converts |
| | isomerase | l-arabinose |
| | | to l-ribulose |
| DTE | d-tagatose | Converts l-ribulose |
| | epimerase | to l-xylulose |

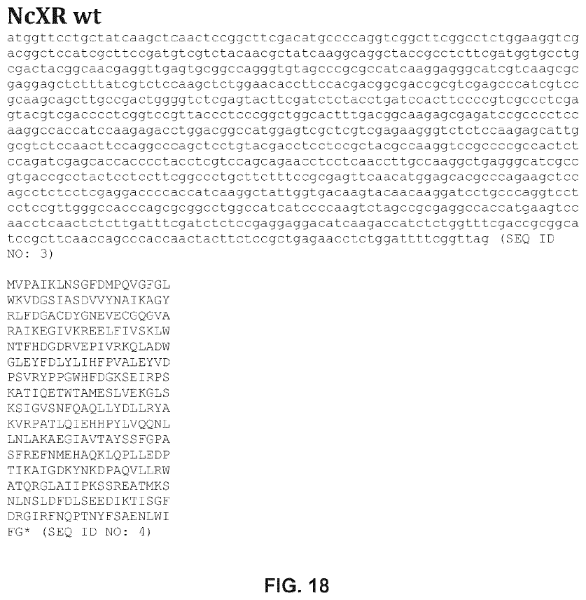

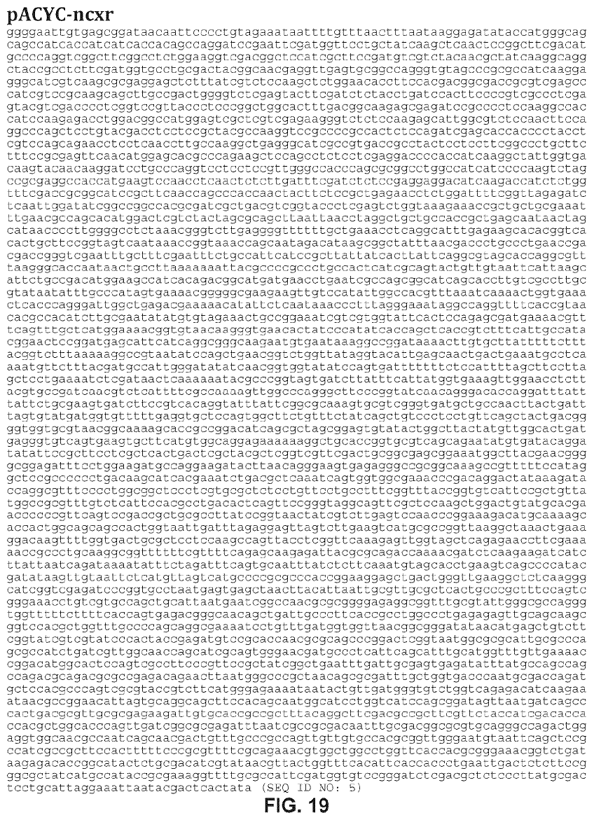

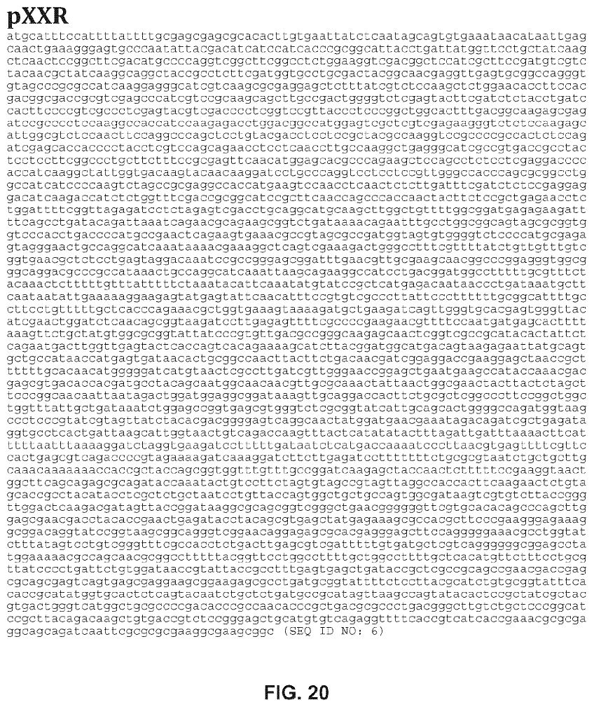

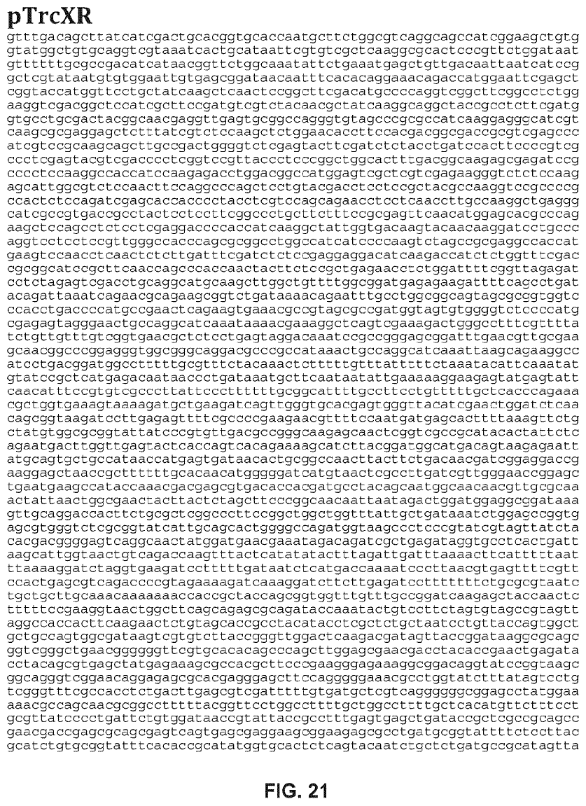

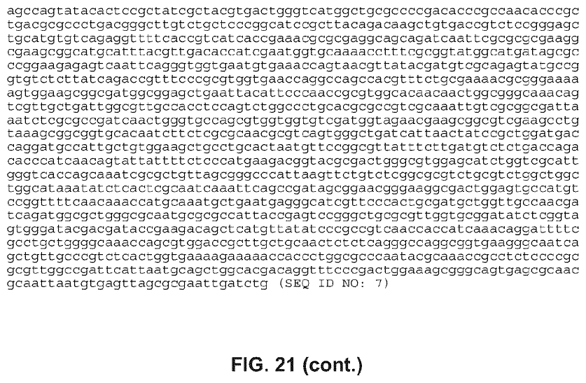

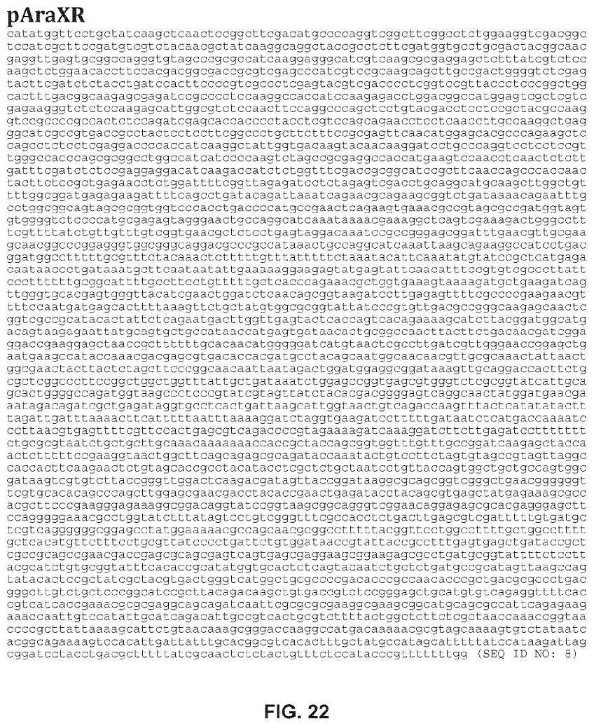

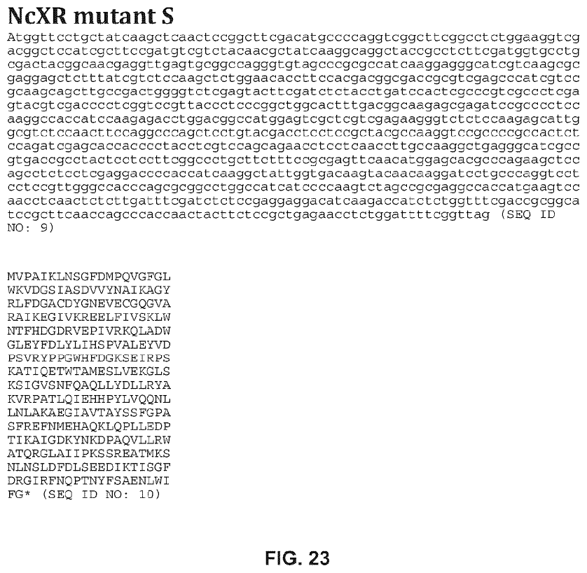

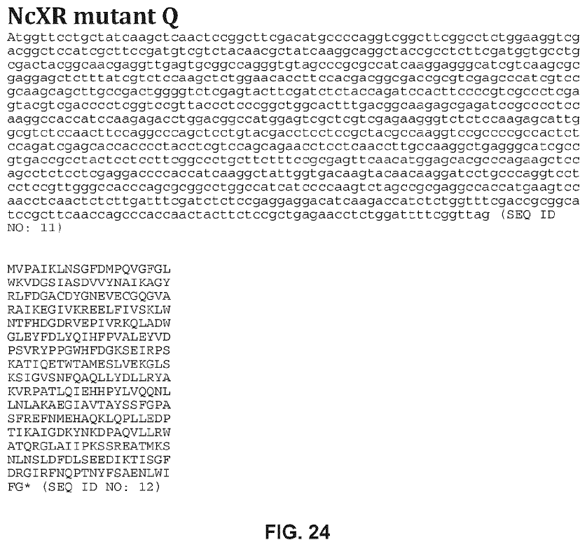

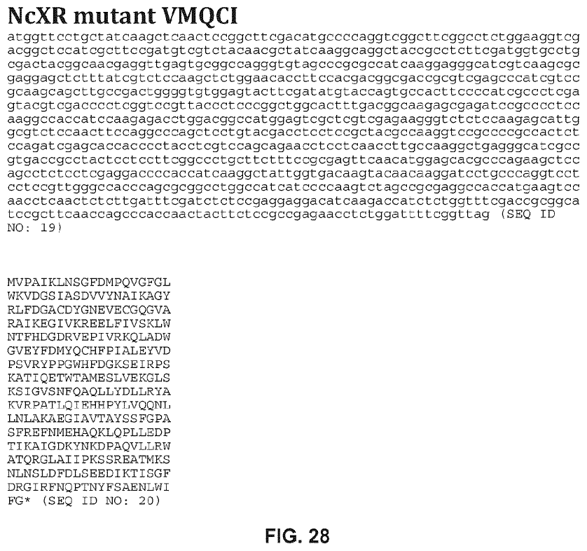

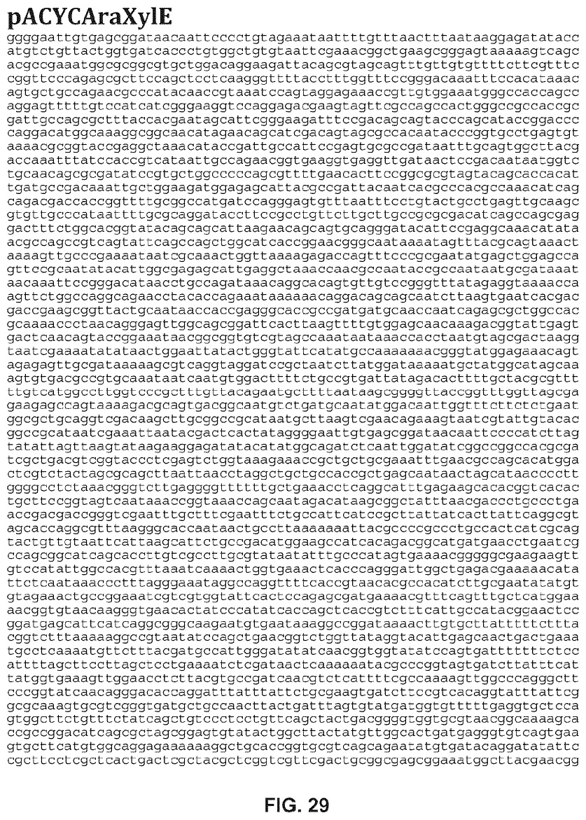

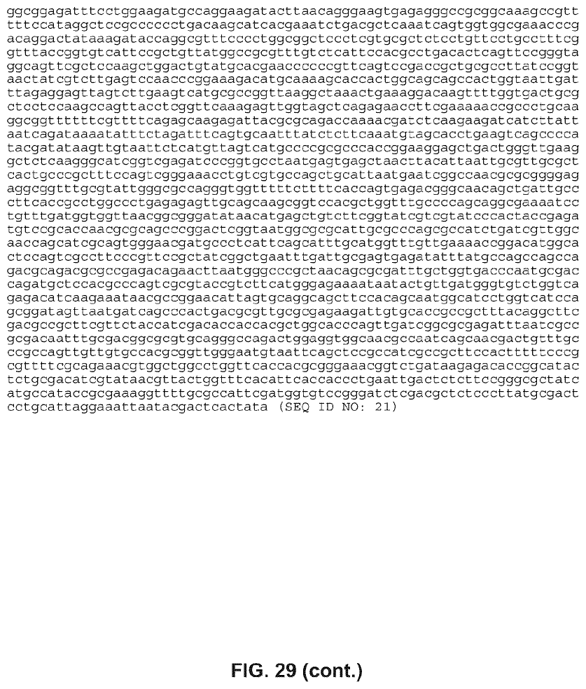

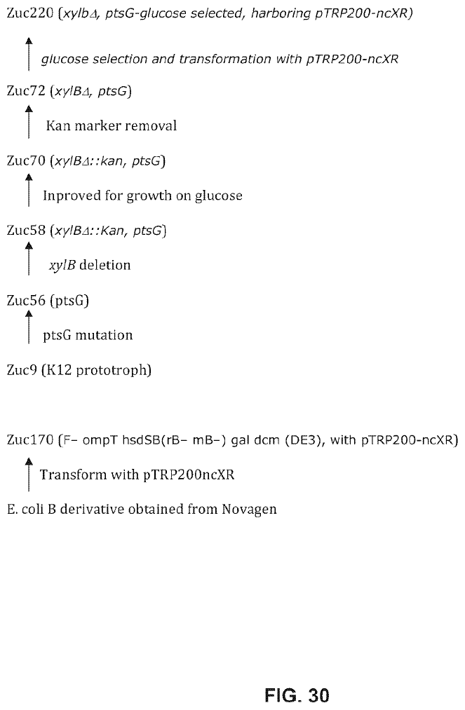

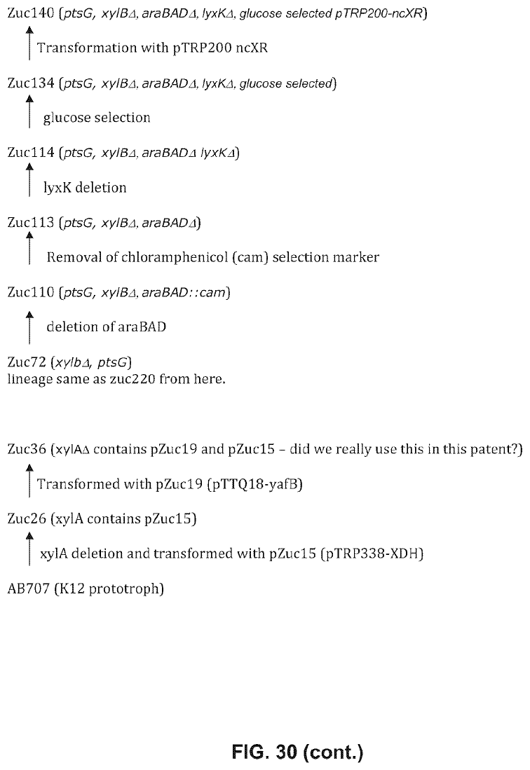

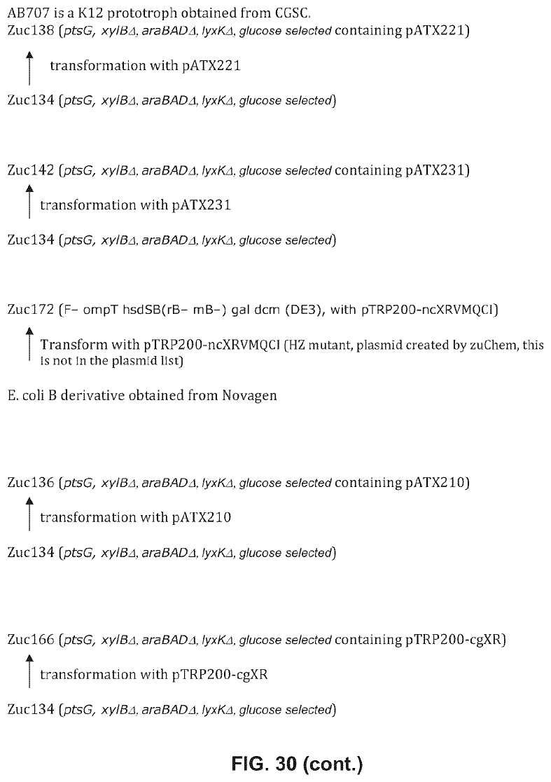

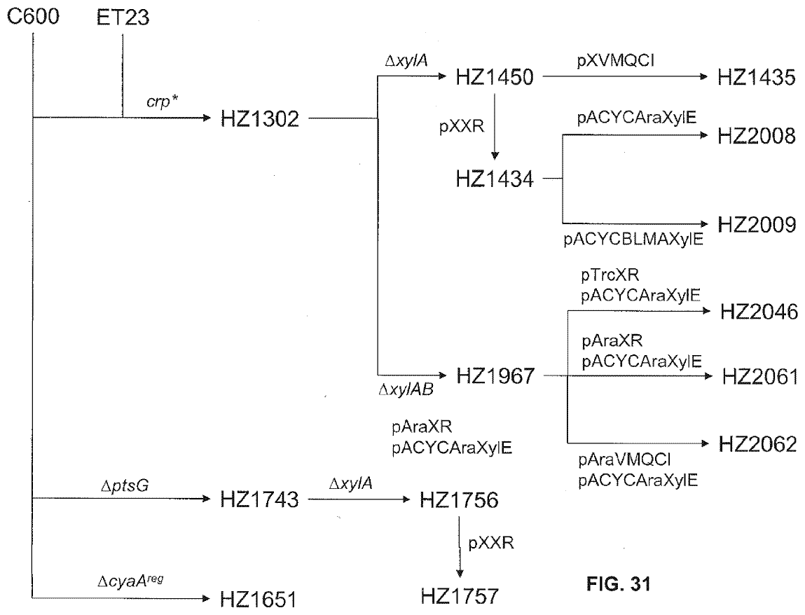

TABLE 1
Strains and plasmids.

| Name | Relevant characteristics | Source/Comments | SEQ |
|---|---|---|---|
| Plasmids | | | |
| pTrc99A | Amp, pBR322-derived plasmid | Amersham Pharmacia | |
| pACYCDuet | Cm, p15A-derived plasmid | Novagen | |
| pACYC-nexr | template for XR | Nair and Zhao, 2008 | (FIG. 18) |
| pACYC-VMQCI | template for XR mutant VMQCI | Nair and Zhao, 2008 | |
| pTKXb-xdharaB′ | Km, Source of BLMA promoter | Nair and Zhao, 2008 | |
| pRW2-ptdh | Km, Source of AraBAD promoter | Johannes et al., 2005 | |
| pXXR | pTrc99A with XR under XylA promoter | Present disclosure | (FIG. 20) |
| pXVMQCI | pTrc99A with VMQCI under XylA promoter | Present disclosure | (FIG. 28) |
| pAraXR | pTrc99A with XR under AraBAD promoter | Present disclosure | (FIG. 22) |
| pAraVMQCI | pTrc99A with VMQCI under AraBAD | Present disclosure | |
| | promoter | | |
| pTrcXR | pTrc99A with XR under Trc promoter | Present disclosure | (FIG. 21) |
| pTrcVMQCI | pTrc99A with VMQCI under Trc promoter | Present disclosure | |
| pACYCBLMAXylE | pACYCDuet with xylE under BLMA | Present disclosure | |
| | promoter | | |
| pACYCAraXylE | pACYCDuet with xylE under AraBAD | Present disclosure | (FIG. 29) |
| | promoter | | |
| pCP20 | | created by Paul Taylor | |
| pTRP338 | pTRP200-pLG338 derivative | | |
| pTRP200 NcXR | Neurospora crassa xylose reductase. NcXR | Present disclosure | |
| | from 7381553. | | |
| pTRP200 CgXR | Chaetomium globosum xylose reductase | Present disclosure | |
| pZUC035 | T. resei (XDH) E. coli (XI) | Taylor patent | |
| pZUC036 | T. resei (XDH) E. coli (XI) | Taylor patent | |
| pZUC052 | T. resei (XDH) E. coli (XI-mutant) | Present disclosure | |
| pATX210 | RtdE (R. radiobacter)/alxR (A. | Sakaibara patent | |
| | monospora)/araA (E. coli) | | |
| pATX215 | RtdE (R. radiobacter)/alxR (A. | Present disclosure | |
| | monospora)/araA (E. coli). pATX210 | | |
| | derivative with additional arabinose BAD | | |
| | promote | | |
| pATX221 | RtdE (R. radiobacter)/alxR (A. | Present disclosure | |
| | monospora)/araA (E. coli)/XR (N. crassa). | | |
| | combines XR with pATX210 ara pathway | | |
| pATX231 | RtdE (R. radiobacter)/alxR (A. | Present disclosure | |
| | monospora)/araA (E. coli)/T. resei (XDH)/E. | | |
| | coli (XI). combines XI/XDH with pATX210 | | |
| | pathway (same orientation of genes) | | |
| pATX231B | RtdE (R. radiobacter)/alxR (A. | Present disclosure | |
| | monospora)/araA (E. coli)/T. resei (XDH)/E. | | |
| | coli (XI). combines XI/XDH with pATX210 | | |
| | pathway (opposite orientation of XI XDH | | |
| | genes) | | |
| Strains | | | |
| MG1655 | gDNA template for XylA promoter and xylE | ATCC 700926 | |
| C600 | F tonA21 thi-1 thr-1 leuB6 lacY1 glnV44 | CGSC, Yale | |
| | rfbC1 fhuA1 χ | University | |
| ET23 | source of crp*::Tn10 | Eppler and Boos, 1999 | |
| HZ1302 | C600 crp*::Tn10 | Present disclosure | |
| HZ1743 | C600 DptsG::FRT | Present disclosure | |
| HZ1651 | C600 DcyaA^{regul}::cat | Present disclosure | |
| HZ1450 | HZ1302 DxylA::FRT | Present disclosure | |
| HZ1967 | HZ1302 DxylAB::FRT | Present disclosure | |
| HZ1756 | HZ1743 DxylA::FRT | Present disclosure | |
| HZ1434 | HZ1450 with pXXR | Present disclosure | |
| HZ1435 | HZ1450 with pXVMQCI | Present disclosure | |
| HZ1757 | HZ1756 with pXXR | Present disclosure | |
| HZ2008 | HZ1450 with pXXR & pACYCAraXylE | Present disclosure | |
| HZ2009 | HZ1450 with pXXR & pACYCBLMAXylE | Present disclosure | |
| HZ2046 | HZ1967 with pTrcXR & pACYCAraXylE | Present disclosure | |
| HZ2061 | HZ1967 with pAraXR & pACYCAraXylE | Present disclosure | |
| HZ2062 | HZ1967 with pAraVMQCI & | Present disclosure | |
| | pACYCAraXylE | | |
| DH5a | | | |
| AB707 | | | |
| ZUC036 | | | |
| ZUC134 | ptsG, xylBD, araBADD, lyxKD, glucose | Present disclosure | |
| | selected (parent is AB707 K12 prototroph) | | |
| ZUC136 | ptsG, xylBD, araBADD, lyxKD, glucose | Present disclosure | |
| | selected contains pATX210 in ZUC134) | | |
| ZUC138 | | | |
| ZUC142 | | | |
| ZUC140 | ptsG, xylBD, araBADD, lyxKD, glucose | Present disclosure | |
| | selected contains pTRP200-ncXR in ZUC134 | | |
| ZUC166 | ptsG, xylBD, araBADD, lyxKD, glucose | Present disclosure | |
| | selected contains pTRP200-CgXR in ZUC134 | | |
| ZUC170 | F− ompT hsdSB(rB− mB−) gal dem (DE3) | Present disclosure | |
| | contains pTRP200-NCXR (E. coli B—BL21 | | |
| | derivative) | | |
| ZUC172 | | | |
| ZUC220 | xylbD, ptsG-glucose selected (AB707 K12 | Present disclosure | |
| | prototroph derivative) | | |

### PUBLICATIONS

The following documents are incorporated by reference to the extent they relate to or describe materials or methods disclosed herein. Specific locations in publications cited appear in the specification.
Akinterinwa, O., Cirino, P. C., 2009. Heterologous expression of D-xylulokinase from Pichia stipitis enables high levels of xylitol production by engineered Escherichia coli growing on xylose. Metab. Eng. 11, 48-55.
Cirino, P. C., et al., 2006. Engineering Escherichia coli for xylitol production from glucose-xylose mixtures. Biotech. Bioeng. 95, 1167-1176.
Datsenko, K. A., Wanner, B. L., 2000. One-step inactivation of chromosomal genes in Escherichia coli K-12 using PCR products. Proc. Natl. Acad. Sci. USA. 97, 6640-6645.
Eiteman, M. A., Altman, E., 2006. Overcoming acetate in Escherichia coli recombinant protein fermentations. Trends Biotechnol. 24, 530-536.
Eppler, T., Boos, W., 1999. Glycerol-3-phosphate-mediated repression of malT in Escherichia co/i does not require metabolism, depends on enzyme IIA(Glc) and is mediated by cAMP levels. Mol. Microbiol. 33, 1221-1231.
Johannes, T. W., et al., 2005. Directed evolution of a thermostable phosphite dehydrogenase for NAD(P)H regeneration. Appl. Environ. Microb. 71, 5728-5734.
Karimova, G., et al., 2004. Relief of catabolite repression in a cAMP-independent catabolite gene activator mutant of Escherichia coli. Res. Microbiol. 155, 76-79.
Kim, Y. W., et al., 2003. Directed evolution of Thermus maltogenic amylase toward enhanced thermal resistance. Appl. Environ. Microb. 69, 4866-4874.
Lindsay, S. E., et al., 1995. Improved strains of recombinant Escherichia coli for ethanol production from sugar mixtures. Appl. Environ. Microb. 43, 70-5.
Miller, J. H., 1992. A short course in bacterial genetics: a laboratory manual and handbook for Escherichia coli and related bacteria. Cold Spring Harbor Laboratory Press, Plainview, N.Y.
Nair, N. U., Zhao, H., 2008. Evolution in reverse: engineering a D-xylose-specific xylose reductase. Chembiochem. 9, 1213-5.
Nichols, N. N., et al., 2001. Use of catabolite repression mutants for fermentation of sugar mixtures to ethanol. Appl. Microbial. Biotechnol. 56, 120-5.
Zha, W. J., et al., 2008. Exploiting genetic diversity by directed evolution: molecular breeding of type III polyketide synthases improves productivity. Mol. Biosyst. 4, 246-248.

1. A method to produce xylitol from a mixture of hemicellulosic sugars.

2. The method to produce xylitol of claim 1, comprising converting xylose alone to xylitol by the action of a xylose reductase.

3. The method to produce xylitol of claim 1, comprising conversion of L-arabinose to xylitol and reducing xylose.

4. The method to produce xylitol of claim 1, comprising reducing D-xylose and metabolizing arabinose.

5. A microorganism capable of converting a mixture of sugars, wherein the sugars are selected from the group consisting of xylose, arabinose, and combinations thereof, and wherein conversion is by fermentation to xylitol with little or no arabitol present in the final fermentation broth.

6. The microorganism of claim 5, wherein the microorganism is an E. coli strain that efficiently produces xylitol from D-xylose, using a mutant xylose reductase (XR) designated VMQCI and a genetically engineered E. coli that utilizes L-arabinose as a carbon source, thereby decreasing L-arabinitol production, and wherein xylitol is produced at a purity of approximately 100% from an equivalent mixture of D-xylose, L-arabinose, and D-glucose, and wherein there is a minimal production of L-arabinitol byproduct.

7. The microorganism of claim 6, wherein the E. coli strain is designated HZ 1434.

8. The microorganism of claim 5, wherein arabitol is less than 10% of the final mixture of polyol products produced.

9. The microorganism of claim 5, wherein arabitol is less than 5% of the final mixture of polyol products produced.

10. The microorganism of claim 5, wherein an initial ratio of xylose:arabinose is greater than 1:1.

11. The microorganism of claim 5, wherein an initial ratio of xylose:arabinose is greater than 2:1.

12. The microorganism of claim 5, wherein the microorganism is an E. coli strain designated ZUC220.

13. The microorganism of claim 5, wherein the microorganism is an E. coli strain designated ZUC 170.

14. A bioprocess for converting a mixture of sugars to xylitol, wherein the mixture includes sugars selected from the group consisting of xylose, arabinose, and combinations thereof, and wherein xylitol is produced with little or no arabinitol present in the final fermentation broth.

15. The bioprocess of claim 14, wherein arabinitol is less than 10% of the final mixture of polyol products produced.


SEQUENCE LISTING (21 sequences)

SEQ ID NO: 1 - protein, 322 aa, Chaetomium globosum
MAPVIKLNSGYDMPQVGFGLWKVDNAVASDVVYNAIKAGYRLFDGACDYGNEVECGQGVARAISEGIVKREDLFIVSKLWNTFHDAERVEPIVKKQLADWGIEYFDLYLIHFPVALEWVDPAVRYPPGWHYDGKEEIRPSKATIQETWTALESLVSKGLSKSIGISNFQAQLIYDLLRYAKIRPATLQVEHHPYLVQQELINLAKREGIAVTAYSSFGPASFKEFNMKHADALAPLIEDETIKKIAAKHNRPASQVLLRWATQRGLAIIPKSTRPQIMAENFQSIDFDLSEEDIATISAFDRGIRFNQPSNYFPTELLWIFG

SEQ ID NO: 2 - DNA, 969 bp, synthetic construct
  misc_feature 1..969: note = Artificial Sequence
  source 1..969: mol_type = other DNA
atggcgccggtgattaaactgaacagcggctatgatatgccgcaggtgggctttggcctgtggaaagtggataacgcggtggcgagcgatgtggtgtataacgcgattaaagcgggctatcgtctgtttgatggcgcgtgcgattatggcaacgaagtggaatgcggccagggtgtggcgcgtgccatcagcgaaggcattgtgaaacgtgaggacctgttcattgtgagcaaactgtggaacacctttcatgatgcggaacgtgtggaaccgattgtgaaaaaacagctggccgattggggcattgaatatttcgatctgtatctgatccattttccggtggcgctggaatgggttgatccggcggtgcgttatccgccgggttggcattatgatggcaaagaagaaattcgtccgagcaaagcgaccattcaggaaacctggaccgcgctggaaagcctggtgagcaaaggcctgagcaaaagcattggcattagcaactttcaggcgcagctgatttatgatctgctgcgctatgcgaaaattcgtccggcgaccctgcaggtggaacatcatccgtatctggtgcagcaggaactgattaacctggccaaacgtgaaggcattgcggtgaccgcgtatagcagctttggtccggccagctttaaagaatttaacatgaaacatgcggatgcgctggccccgctgattgaagatgaaaccatcaaaaaaatcgcggcgaaacataaccgtccggcgagccaggttctgctgcgttgggcgacccagcgtggcctggccattattccgaaaagcacccgtccgcagattatggcggaaaactttcagagcatcgattttgatctgagcgaagaagatattgcgaccattagcgcgtttgatcgtggcattcgttttaaccagccgagcaactattttccgaccgaactgctgtggatttttggctaa

SEQ ID NO: 3 - DNA, 969 bp, Neurospora crassa
  source 1..969: mol_type = other DNA
atggttcctgctatcaagctcaactccggcttcgacatgccccaggtcggcttcggcctctggaaggtcgacggctccatcgcttccgatgtcgtctacaacgctatcaaggcaggctaccgcctcttcgatggtgcctgcgactacggcaacgaggttgagtgcggccagggtgtagcccgcgccatcaaggagggcatcgtcaagcgcgaggagctctttatcgtctccaagctctggaacaccttccacgacggcgaccgcgtcgagcccatcgtccgcaagcagcttgccgactggggtctcgagtacttcgatctctacctgatccacttccccgtcgccctcgagtacgtcgacccctcggtccgttaccctcccggctggcactttgacggcaagagcgagatccgcccctccaaggccaccatccaagagacctggacggccatggagtcgctcgtcgagaagggtctctccaagagcattggcgtctccaacttccaggcccagctcctgtacgacctcctccgctacgccaaggtccgccccgccactctccagatcgagcaccacccctacctcgtccagcagaacctcctcaaccttgccaaggctgagggcatcgccgtgaccgcctactcctccttcggccctgcttctttccgcgagttcaacatggagcacgcccagaagctccagcctctcctcgaggaccccaccatcaaggctattggtgacaagtacaacaaggatcctgcccaggtcctcctccgttgggccacccagcgcggcctggccatcatccccaagtctagccgcgaggccaccatgaagtccaacctcaactctcttgatttcgatctctccgaggaggacatcaagaccatctctggtttcgaccgcggcatccgcttcaaccagcccaccaactacttctccgctgagaacctctggattttcggttag

SEQ ID NO: 4 - protein, 322 aa, Neurospora crassa
MVPAIKLNSGFDMPQVGFGLWKVDGSIASDVVYNAIKAGYRLFDGACDYGNEVECGQGVARAIKEGIVKREELFIVSKLWNTFHDGDRVEPIVRKQLADWGLEYFDLYLIHFPVALEYVDPSVRYPPGWHFDGKSEIRPSKATIQETWTAMESLVEKGLSKSIGVSNFQAQLLYDLLRYAKVRPATLQIEHHPYLVQQNLLNLAKAEGIAVTAYSSFGPASFREFNMEHAQKLQPLLEDPTIKAIGDKYNKDPAQVLLRWATQRGLAIIPKSSREATMKSNLNSLDFDLSEEDIKTISGFDRGIRFNQPTNYFSAENLWIFG

SEQ ID NO: 5 - DNA, 4791 bp, synthetic construct
  misc_feature 1..4791: note = Artificial Sequence
  source 1..4791: mol_type = other DNA
ggggaattgtgagcggataacaattcccctgtagaaataattttgtttaactttaataaggagatataccatgggcagcagccatcaccatcatcaccacagccaggatccgaattcgatggttcctgctatcaagctcaactccggcttcgacatgccccaggtcggcttcggcctctggaaggtcgacggctccatcgcttccgatgtcgtctacaacgctatcaaggcaggctaccgcctcttcgatggtgcctgcgactacggcaacgaggttgagtgcggccagggtgtagcccgcgccatcaaggagggcatcgtcaagcgcgaggagctctttatcgtctccaagctctggaacaccttccacgacggcgaccgcgtcgagcccatcgtccgcaagcagcttgccgactggggtctcgagtacttcgatctctacctgatccacttccccgtcgccctcgagtacgtcgacccctcggtccgttaccctcccggctggcactttgacggcaagagcgagatccgcccctccaaggccaccatccaagagacctggacggccatggagtcgctcgtcgagaagggtctctccaagagcattggcgtctccaacttccaggcccagctcctgtacgacctcctccgctacgccaaggtccgccccgccactctccagatcgagcaccacccctacctcgtccagcagaacctcctcaaccttgccaaggctgagggcatcgccgtgaccgcctactcctccttcggccctgcttctttccgcgagttcaacatggagcacgcccagaagctccagcctctcctcgaggaccccaccatcaaggctattggtgacaagtacaacaaggatcctgcccaggtcctcctccgttgggccacccagcgcggcctggccatcatccccaagtctagccgcgaggccaccatgaagtccaacctcaactctcttgatttcgatctctccgaggaggacatcaagaccatctctggtttcgaccgcggcatccgcttcaaccagcccaccaactacttctccgctgagaacctctggattttcggttagagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 6 - DNA, 3780 bp, synthetic construct
  misc_feature 1..3780: note = Artificial Sequence
  source 1..3780: mol_type = other DNA
atgcatttccattttattttgcgagcgagcgcacacttgtgaattatctcaatagcagtgtgaaataacataattgagcaactgaaagggagtgcccaatattacgacatcatccatcacccgcggcattacctgattatggttcctgctatcaagctcaactccggcttcgacatgccccaggtcggcttcggcctctggaaggtcgacggctccatcgcttccgatgtcgtctacaacgctatcaaggcaggctaccgcctcttcgatggtgcctgcgactacggcaacgaggttgagtgcggccagggtgtagcccgcgccatcaaggagggcatcgtcaagcgcgaggagctctttatcgtctccaagctctggaacaccttccacgacggcgaccgcgtcgagcccatcgtccgcaagcagcttgccgactggggtctcgagtacttcgatctctacctgatccacttccccgtcgccctcgagtacgtcgacccctcggtccgttaccctcccggctggcactttgacggcaagagcgagatccgcccctccaaggccaccatccaagagacctggacggccatggagtcgctcgtcgagaagggtctctccaagagcattggcgtctccaacttccaggcccagctcctgtacgacctcctccgctacgccaaggtccgccccgccactctccagatcgagcaccacccctacctcgtccagcagaacctcctcaaccttgccaaggctgagggcatcgccgtgaccgcctactcctccttcggccctgcttctttccgcgagttcaacatggagcacgcccagaagctccagcctctcctcgaggaccccaccatcaaggctattggtgacaagtacaacaaggatcctgcccaggtcctcctccgttgggccacccagcgcggcctggccatcatccccaagtctagccgcgaggccaccatgaagtccaacctcaactctcttgatttcgatctctccgaggaggacatcaagaccatctctggtttcgaccgcggcatccgcttcaaccagcccaccaactacttctccgctgagaacctctggattttcggttagagatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggc

SEQ ID NO: 7 - DNA, 5142 bp, synthetic construct
  misc_feature 1..5142: note = Artificial Sequence
  source 1..5142: mol_type = other DNA
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatggaattcgagctcggtaccatggttcctgctatcaagctcaactccggcttcgacatgccccaggtcggcttcggcctctggaaggtcgacggctccatcgcttccgatgtcgtctacaacgctatcaaggcaggctaccgcctcttcgatggtgcctgcgactacggcaacgaggttgagtgcggccagggtgtagcccgcgccatcaaggagggcatcgtcaagcgcgaggagctctttatcgtctccaagctctggaacaccttccacgacggcgaccgcgtcgagcccatcgtccgcaagcagcttgccgactggggtctcgagtacttcgatctctacctgatccacttccccgtcgccctcgagtacgtcgacccctcggtccgttaccctcccggctggcactttgacggcaagagcgagatccgcccctccaaggccaccatccaagagacctggacggccatggagtcgctcgtcgagaagggtctctccaagagcattggcgtctccaacttccaggcccagctcctgtacgacctcctccgctacgccaaggtccgccccgccactctccagatcgagcaccacccctacctcgtccagcagaacctcctcaaccttgccaaggctgagggcatcgccgtgaccgcctactcctccttcggccctgcttctttccgcgagttcaacatggagcacgcccagaagctccagcctctcctcgaggaccccaccatcaaggctattggtgacaagtacaacaaggatcctgcccaggtcctcctccgttgggccacccagcgcggcctggccatcatccccaagtctagccgcgaggccaccatgaagtccaacctcaactctcttgatttcgatctctccgaggaggacatcaagaccatctctggtttcgaccgcggcatccgcttcaaccagcccaccaactacttctccgctgagaacctctggattttcggttagagatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 8 - DNA, 3962 bp, synthetic construct
  misc_feature 1..3962: note = Artificial Sequence
  source 1..3962: mol_type = other DNA
catatggttcctgctatcaagctcaactccggcttcgacatgccccaggtcggcttcggcctctggaaggtcgacggctccatcgcttccgatgtcgtctacaacgctatcaaggcaggctaccgcctcttcgatggtgcctgcgactacggcaacgaggttgagtgcggccagggtgtagcccgcgccatcaaggagggcatcgtcaagcgcgaggagctctttatcgtctccaagctctggaacaccttccacgacggcgaccgcgtcgagcccatcgtccgcaagcagcttgccgactggggtctcgagtacttcgatctctacctgatccacttccccgtcgccctcgagtacgtcgacccctcggtccgttaccctcccggctggcactttgacggcaagagcgagatccgcccctccaaggccaccatccaagagacctggacggccatggagtcgctcgtcgagaagggtctctccaagagcattggcgtctccaacttccaggcccagctcctgtacgacctcctccgctacgccaaggtccgccccgccactctccagatcgagcaccacccctacctcgtccagcagaacctcctcaaccttgccaaggctgagggcatcgccgtgaccgcctactcctccttcggccctgcttctttccgcgagttcaacatggagcacgcccagaagctccagcctctcctcgaggaccccaccatcaaggctattggtgacaagtacaacaaggatcctgcccaggtcctcctccgttgggccacccagcgcggcctggccatcatccccaagtctagccgcgaggccaccatgaagtccaacctcaactctcttgatttcgatctctccgaggaggacatcaagaccatctctggtttcgaccgcggcatccgcttcaaccagcccaccaactacttctccgctgagaacctctggattttcggttagagatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcagcgccattcagagaagaaaccaattgtccatattgcatcagacattgccgtcactgcgtcttttactggctcttctcgctaaccaaaccggtaaccccgcttattaaaagcattctgtaacaaagcgggaccaaggccatgacaaaaacgcgtagcaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgg

SEQ ID NO: 9 - DNA, 969 bp, Neurospora crassa
  source 1..969: mol_type = other DNA
atggttcctgctatcaagctcaactccggcttcgacatgccccaggtcggcttcggcctctggaaggtcgacggctccatcgcttccgatgtcgtctacaacgctatcaaggcaggctaccgcctcttcgatggtgcctgcgactacggcaacgaggttgagtgcggccagggtgtagcccgcgccatcaaggagggcatcgtcaagcgcgaggagctctttatcgtctccaagctctggaacaccttccacgacggcgaccgcgtcgagcccatcgtccgcaagcagcttgccgactggggtctcgagtacttcgatctctacctgatccactcgcccgtcgccctcgagtacgtcgacccctcggtccgttaccctcccggctggcactttgacggcaagagcgagatccgcccctccaaggccaccatccaagagacctggacggccatggagtcgctcgtcgagaagggtctctccaagagcattggcgtctccaacttccaggcccagctcctgtacgacctcctccgctacgccaaggtccgccccgccactctccagatcgagcaccacccctacctcgtccagcagaacctcctcaaccttgccaaggctgagggcatcgccgtgaccgcctactcctccttcggccctgcttctttccgcgagttcaacatggagcacgcccagaagctccagcctctcctcgaggaccccaccatcaaggctattggtgacaagtacaacaaggatcctgcccaggtcctcctccgttgggccacccagcgcggcctggccatcatccccaagtctagccgcgaggccaccatgaagtccaacctcaactctcttgatttcgatctctccgaggaggacatcaagaccatctctggtttcgaccgcggcatccgcttcaaccagcccaccaactacttctccgctgagaacctctggattttcggttag

SEQ ID NO: 10 - protein, 322 aa, Neurospora crassa
MVPAIKLNSGFDMPQVGFGLWKVDGSIASDVVYNAIKAGYRLFDGACDYGNEVECGQGVARAIKEGIVKREELFIVSKLWNTFHDGDRVEPIVRKQLADWGLEYFDLYLIHSPVALEYVDPSVRYPPGWHFDGKSEIRPSKATIQETWTAMESLVEKGLSKSIGVSNFQAQLLYDLLRYAKVRPATLQIEHHPYLVQQNLLNLAKAEGIAVTAYSSFGPASFREFNMEHAQKLQPLLEDPTIKAIGDKYNKDPAQVLLRWATQRGLAIIPKSSREATMKSNLNSLDFDLSEEDIKTISGFDRGIRFNQPTNYFSAENLWIFG

SEQ ID NO: 11 - DNA, 969 bp, Neurospora crassa
  source 1..969: mol_type = other DNA
atggttcctgctatcaagctcaactccggcttcgacatgccccaggtcggcttcggcctctggaaggtcgacggctccatcgcttccgatgtcgtctacaacgctatcaaggcaggctaccgcctcttcgatggtgcctgcgactacggcaacgaggttgagtgcggccagggtgtagcccgcgccatcaaggagggcatcgtcaagcgcgaggagctctttatcgtctccaagctctggaacaccttccacgacggcgaccgcgtcgagcccatcgtccgcaagcagcttgccgactggggtctcgagtacttcgatctctaccagatccacttccccgtcgccctcgagtacgtcgacccctcggtccgttaccctcccggctggcactttgacggcaagagcgagatccgcccctccaaggccaccatccaagagacctggacggccatggagtcgctcgtcgagaagggtctctccaagagcattggcgtctccaacttccaggcccagctcctgtacgacctcctccgctacgccaaggtccgccccgccactctccagatcgagcaccacccctacctcgtccagcagaacctcctcaaccttgccaaggctgagggcatcgccgtgaccgcctactcctccttcggccctgcttctttccgcgagttcaacatggagcacgcccagaagctccagcctctcctcgaggaccccaccatcaaggctattggtgacaagtacaacaaggatcctgcccaggtcctcctccgttgggccacccagcgcggcctggccatcatccccaagtctagccgcgaggccaccatgaagtccaacctcaactctcttgatttcgatctctccgaggaggacatcaagaccatctctggtttcgaccgcggcatccgcttcaaccagcccaccaactacttctccgctgagaacctctggattttcggttag

SEQ ID NO: 12 - protein, 322 aa, Neurospora crassa
MVPAIKLNSGFDMPQVGFGLWKVDGSIASDVVYNAIKAGYRLFDGACDYGNEVECGQGVARAIKEGIVKREELFIVSKLWNTFHDGDRVEPIVRKQLADWGLEYFDLYQIHFPVALEYVDPSVRYPPGWHFDGKSEIRPSKATIQETWTAMESLVEKGLSKSIGVSNFQAQLLYDLLRYAKVRPATLQIEHHPYLVQQNLLNLAKAEGIAVTAYSSFGPASFREFNMEHAQKLQPLLEDPTIKAIGDKYNKDPAQVLLRWATQRGLAIIPKSSREATMKSNLNSLDFDLSEEDIKTISGFDRGIRFNQPTNYFSAENLWIFG

SEQ ID NO: 13 - DNA, 969 bp, Neurospora crassa
  source 1..969: mol_type = other DNA
atggttcctgctatcaagctcaactccggcttcgacatgccccaggtcggcttcggcctctggaaggtcgacggctccatcgcttccgatgtcgtctacaacgctatcaaggcaggctaccgcctcttcgatggtgcctgcgactacggcaacgaggttgagtgcggccagggtgtagcccgcgccatcaaggagggcatcgtcaagcgcgaggagctctttatcgtctccaagctctggaacaccttccacgacggcgaccgcgtcgagcccatcgtccgcaagcagcttgccgactggggtctcgagtacttcgatctctaccagtgccacttccccgtcgccctcgagtacgtcgacccctcggtccgttaccctcccggctggcactttgacggcaagagcgagatccgcccctccaaggccaccatccaagagacctggacggccatggagtcgctcgtcgagaagggtctctccaagagcattggcgtctccaacttccaggcccagctcctgtacgacctcctccgctacgccaaggtccgccccgccactctccagatcgagcaccacccctacctcgtccagcagaacctcctcaaccttgccaaggctgagggcatcgccgtgaccgcctactcctccttcggccctgcttctttccgcgagttcaacatggagcacgcccagaagctccagcctctcctcgaggaccccaccatcaaggctattggtgacaagtacaacaaggatcctgcccaggtcctcctccgttgggccacccagcgcggcctggccatcatccccaagtctagccgcgaggccaccatgaagtccaacctcaactctcttgatttcgatctctccgaggaggacatcaagaccatctctggtttcgaccgcggcatccgcttcaaccagcccaccaactacttctccgctgagaacctctggattttcggttag

SEQ ID NO: 14 - protein, 322 aa, Neurospora crassa
MVPAIKLNSGFDMPQVGFGLWKVDGSIASDVVYNAIKAGYRLFDGACDYGNEVECGQGVARAIKEGIVKREELFIVSKLWNTFHDGDRVEPIVRKQLADWGLEYFDLYQCHFPVALEYVDPSVRYPPGWHFDGKSEIRPSKATIQETWTAMESLVEKGLSKSIGVSNFQAQLLYDLLRYAKVRPATLQIEHHPYLVQQNLLNLAKAEGIAVTAYSSFGPASFREFNMEHAQKLQPLLEDPTIKAIGDKYNKDPAQVLLRWATQRGLAIIPKSSREATMKSNLNSLDFDLSEEDIKTISGFDRGIRFNQPTNYFSAENLWIFG

SEQ ID NO: 15 - DNA, 969 bp, Neurospora crassa
  source 1..969: mol_type = other DNA
atggttcctgctatcaagctcaactccggcttcgacatgccccaggtcggcttcggcctctggaaggtcgacggctccatcgcttccgatgtcgtctacaacgctatcaaggcaggctaccgcctcttcgatggtgcctgcgactacggcaacgaggttgagtgcggccagggtgtagcccgcgccatcaaggagggcatcgtcaagcgcgaggagctctttatcgtctccaagctctggaacaccttccacgacggcgaccgcgtcgagcccatcgtccgcaagcagcttgccgactggggtctcgagtacttcgatatgtaccagtgccacttccccgtcgccctcgagtacgtcgacccctcggtccgttaccctcccggctggcactttgacggcaagagcgagatccgcccctccaaggccaccatccaagagacctggacggccatggagtcgctcgtcgagaagggtctctccaagagcattggcgtctccaacttccaggcccagctcctgtacgacctcctccgctacgccaaggtccgccccgccactctccagatcgagcaccacccctacctcgtccagcagaacctcctcaaccttgccaaggctgagggcatcgccgtgaccgcctactcctccttcggccctgcttctttccgcgagttcaacatggagcacgcccagaagctccagcctctcctcgaggaccccaccatcaaggctattggtgacaagtacaacaaggatcctgcccaggtcctcctccgttgggccacccagcgcggcctggccatcatccccaagtctagccgcgaggccaccatgaagtccaacctcaactctcttgatttcgatctctccgaggaggacatcaagaccatctctggtttcgaccgcggcatccgcttcaaccagcccaccaactacttctccgctgagaacctctggattttcggttag

SEQ ID NO: 16 - protein, 322 aa, Neurospora crassa
MVPAIKLNSGFDMPQVGFGLWKVDGSIASDVVYNAIKAGYRLFDGACDYGNEVECGQGVARAIKEGIVKREELFIVSKLWNTFHDGDRVEPIVRKQLADWGLEYFDMYQCHFPVALEYVDPSVRYPPGWHFDGKSEIRPSKATIQETWTAMESLVEKGLSKSIGVSNFQAQLLYDLLRYAKVRPATLQIEHHPYLVQQNLLNLAKAEGIAVTAYSSFGPASFREFNMEHAQKLQPLLEDPTIKAIGDKYNKDPAQVLLRWATQRGLAIIPKSSREATMKSNLNSLDFDLSEEDIKTISGFDRGIRFNQPTNYFSAENLWIFG

SEQ ID NO: 17 - DNA, 969 bp, Neurospora crassa
  source 1..969: mol_type = other DNA
atggttcctgctatcaagctcaactccggcttcgacatgccccaggtcggcttcggcctctggaaggtcgacggctccatcgcttccgatgtcgtctacaacgctatcaaggcaggctaccgcctcttcgatggtgcctgcgactacggcaacgaggttgagtgcggccagggtgtagcccgcgccatcaaggagggcatcgtcaagcgcgaggagctctttatcgtctccaagctctggaacaccttccacgacggcgaccgcgtcgagcccatcgtccgcaagcagcttgccgactggggtctcgagtacttcgatatgtaccagtgccacttccccatcgccctcgagtacgtcgacccctcggtccgttaccctcccggctggcactttgacggcaagagcgagatccgcccctccaaggccaccatccaagagacctggacggccatggagtcgctcgtcgagaagggtctctccaagagcattggcgtctccaacttccaggcccagctcctgtacgacctcctccgctacgccaaggtccgccccgccactctccagatcgagcaccacccctacctcgtccagcagaacctcctcaaccttgccaaggctgagggcatcgccgtgaccgcctactcctccttcggccctgcttctttccgcgagttcaacatggagcacgcccagaagctccagcctctcctcgaggaccccaccatcaaggctattggtgacaagtacaacaaggatcctgcccaggtcctcctccgttgggccacccagcgcggcctggccatcatccccaagtctagccgcgaggccaccatgaagtccaacctcaactctcttgatttcgatctctccgaggaggacatcaagaccatctctggtttcgaccgcggcatccgcttcaaccagcccaccaactacttctccgctgagaacctctggattttcggttag

SEQ ID NO: 18 - protein, 322 aa, Neurospora crassa
MVPAIKLNSGFDMPQVGFGLWKVDGSIASDVVYNAIKAGYRLFDGACDYGNEVECGQGVARAIKEGIVKREELFIVSKLWNTFHDGDRVEPIVRKQLADWGLEYFDMYQCHFPIALEYVDPSVRYPPGWHFDGKSEIRPSKATIQETWTAMESLVEKGLSKSIGVSNFQAQLLYDLLRYAKVRPATLQIEHHPYLVQQNLLNLAKAEGIAVTAYSSFGPASFREFNMEHAQKLQPLLEDPTIKAIGDKYNKDPAQVLLRWATQRGLAIIPKSSREATMKSNLNSLDFDLSEEDIKTISGFDRGIRFNQPTNYFSAENLWIFG

SEQ ID NO: 19 - DNA, 969 bp, Neurospora crassa
  source 1..969: mol_type = other DNA
atggttcctgctatcaagctcaactccggcttcgacatgccccaggtcggcttcggcctctggaaggtcgacggctccatcgcttccgatgtcgtctacaacgctatcaaggcaggctaccgcctcttcgatggtgcctgcgactacggcaacgaggttgagtgcggccagggtgtagcccgcgccatcaaggagggcatcgtcaagcgcgaggagctctttatcgtctccaagctctggaacaccttccacgacggcgaccgcgtcgagcccatcgtccgcaagcagcttgccgactggggtgtggagtacttcgatatgtaccagtgccacttccccatcgccctcgagtacgtcgacccctcggtccgttaccctcccggctggcactttgacggcaagagcgagatccgcccctccaaggccaccatccaagagacctggacggccatggagtcgctcgtcgagaagggtctctccaagagcattggcgtctccaacttccaggcccagctcctgtacgacctcctccgctacgccaaggtccgccccgccactctccagatcgagcaccacccctacctcgtccagcagaacctcctcaaccttgccaaggctgagggcatcgccgtgaccgcctactcctccttcggccctgcttctttccgcgagttcaacatggagcacgcccagaagctccagcctctcctcgaggaccccaccatcaaggctattggtgacaagtacaacaaggatcctgcccaggtcctcctccgttgggccacccagcgcggcctggccatcatccccaagtctagccgcgaggccaccatgaagtccaacctcaactctcttgatttcgatctctccgaggaggacatcaagaccatctctggtttcgaccgcggcatccgcttcaaccagcccaccaactacttctccgccgagaacctctggattttcggttag

SEQ ID NO: 20 - protein, 322 aa, Neurospora crassa
MVPAIKLNSGFDMPQVGFGLWKVDGSIASDVVYNAIKAGYRLFDGACDYGNEVECGQGVARAIKEGIVKREELFIVSKLWNTFHDGDRVEPIVRKQLADWGVEYFDMYQCHFPIALEYVDPSVRYPPGWHFDGKSEIRPSKATIQETWTAMESLVEKGLSKSIGVSNFQAQLLYDLLRYAKVRPATLQIEHHPYLVQQNLLNLAKAEGIAVTAYSSFGPASFREFNMEHAQKLQPLLEDPTIKAIGDKYNKDPAQVLLRWATQRGLAIIPKSSREATMKSNLNSLDFDLSEEDIKTISGFDRGIRFNQPTNYFSAENLWIFG

SEQ ID NO: 21 - DNA, 5842 bp, synthetic construct
  misc_feature 1..5842: note = Artificial Sequence
  source 1..5842: mol_type = other DNA
ggggaattgtgagcggataacaattcccctgtagaaataattttgtttaactttaataaggagatataccatgtctgttactggtgatcaccctgtggctgtgtaattcgaaacggctgaagcgggagtaaaaagtcagcacgccgaaatggcgcggcgtgctggacaggaagattacagcgtagcagtttgttgtgttttcttcgtttccggttcccagagcgcttccagctcctcaagggttttacctttggtttccgggacaaatttccacataaacagtgctgccagaacgcccatacaaccgtaaatccagtaggagaaaccgttgtggaaatgggccaccagccaggagtttttgtccatcatcgggaaggtccaggagacgaagtagttcgccagccactgggccgccaccgcgattgccagcgctttaccacgaatagcattcgggaagatttccgacagcagtacccagcataccggaccccaggacatggcaaaggcggcaacatagaacagcatcgacagtagcgccacaatacccggtgcctgagtgtaaaacgcggtaccgaggctaaacataccgattgccattccgagtgcgccgataatttgcagtggcttacgaccaaatttatccaccgtcataattgccagaacggtgaaggtgaggttgataactccgacaataatggtctgcaacagcgcgatatccgtgctggcccccagcgttttgaacacttccggcgcgtagtacagcaccacattgatgccgacaaattgctggaagatggagagcattacgccgattacaatcacgcccacgccaaacatcagcagacgaccaccggttttgcggccatgatccagggagtgtttaatttcctgtactgcctgagttgcaagcgtgttgcccataattttgcgcaggataccttccgcctgttcttgcttgccgcgcgacatcagccagcgaggactttctggcacggtatacagcagcattaagaacagcagtgcagggatacattccgaggcaaacatataacgccagccgtcagtattcagccagctggcatcaccggaacgggcaataaaatagtttacgcagtaaactaaaagttgcccgaaaataatcgcaaactggttaaaagagaccagtttcccgcgaatatgagctggagccagttccgcaatatacattggcgagagcattgaggctaaaccaacgccaataccgccaataatgcgataaataacaaattccgggacataacctgccagataaacaggcacagtgttgtccgggtttatagaggtaaaaccaagttctggccaggcagaacctacaccagaaataaaaaacaggacagcagcaatcttaagtgaatcacgacgaccgaagcggttactgcaataaccaccgagggcaccgccgatgatgcaaccaatcagagcgctggccacgcaaaaccctaacagggagttggcagcggattcacttaagttttgtggagcaacaaagacggtattgagtgactcaacagtaccggaaataacggcggtgtcgtagccaaataataaaccacctaatgtagcgactaaggtaatcgaaaatatataactggaattatactgggtattcatatgccaaaaaaacgggtatggagaaacagtagagagttgcgataaaaagcgtcaggtaggatccgctaatcttatggataaaaatgctatggcatagcaaagtgtgacgccgtgcaaataatcaatgtggacttttctgccgtgattatagacacttttgctacgcgtttttgtcatggccttggtcccgctttgttacagaatgcttttaataagcggggttaccggtttggttagcgagaagagccagtaaaagacgcagtgacggcaatgtctgatgcaatatggacaattggtttcttctctgaatggcgctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata